(19) United States
(12) Patent Application Publication
(10) Pub. No.: US 2002/0197654 A1
(43) Pub. Date: Dec. 26, 2002

(54) Method for measuring serine palmitoyltransferase in mammalian tissue and use thereof

(76) Inventors: Jill M. Carton, Malvern, PA (US); Michael R. D'Andrea, Cherry Hill, NJ (US); David J. Uhlinger, Flemington, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/100,861

(22) Filed: Mar. 19, 2002

Related U.S. Application Data
(60) Provisional application No. 60/277,252, filed on Mar. 20, 2001.

(51) Int. Cl. G01N 33/567

(52) U.S. Cl. 435/7.21; 424/9.6

(57) ABSTRACT

The present invention is directed to a method for comparatively measuring the level of normal and hyperproliferative serine palmitoyltransferase expression in a mammalian cell and uses thereof.

### CROSS REFERENCE TO RELATED APPLICATIONS

This application claims benefit of provisional application Serial No. 60/277,252, filed Mar. 20, 2001, which is hereby incorporated by reference.

### FIELD OF THE INVENTION

The present invention provides a method for measuring the expression level of serine palmitoyltransferase in a mammalian cell and use thereof. More particularly, the present invention provides a method for comparatively measuring the expression level of serine palmitoyltransferase in a normal and hyperproliferative mammalian cell and uses thereof.

### BACKGROUND OF THE INVENTION

Membrane lipid compositions are highly characteristic of different membranes and can depend on the physiological state of the cell, thus making it important to understand the regulation of these phenomena (Merrill, A. H., Jr., Characterization of serine palmitoyltransferase activity in Chinese hamster ovary cells, Biochimica et Biophysica Acta, 1983, 754, 284-91). Sphingolipids are ubiquitous components of eukaryotic, not prokaryotic, cell membranes. Besides providing structural integrity to cell and organelle membranes, there is emerging evidence for the involvement of sphingolipids in regulating various cellular functions. Sphingolipid metabolic intermediates such as sphingosine, sphingosine-1-phosphate (S-1-P) and ceramide are involved in the regulation of cell growth and differentiation, senescence cell cycle and proliferation apoptosis (D. K. Perry, J. Carton, A. K. Shah, F. Meredith, D. J. Uhlinger and Y. A. Hannun, Serine palmitoyltransferase regulates de novo ceramide generation during etoposide-induced apoptosis, J. Biol. Chem., 2000, 275, 9078-84). Other signaling functions include inhibition of the DAG/PKC pathway, Ca²⁺ mobilization and K⁺ influx within cells. These and other functions of sphingolipids underline the potential importance of sphingolipid metabolism in physiologically important phenomena such as tumor suppression, tissue development, injury and atrophy (reviewed in Y. A. Hannun, Sphingolipid second messengers: Tumor suppressor lipids: Eicosanoids and Other Bioactive Lipids, Cancer, Inflammation and Radiation Injury, 1997, 2; 305-312).

Enzymes regulating sphingolipid metabolism are critical in maintaining cellular bomeostasis and a disruption in their activity can lead to disease. Inhibition of ceramide synthase by fumonisin mycotoxins contaminating animal feeds result in equine leukoencephalomalacia and porcine pulmonary edema. Lowering S-1-P production in TNF-α induced endothelial cells by HDL reduces the expression of adhesion proteins and consequently increases protection against artherosclerosis. Thus, enzymes regulating sphingolipid metabolism are key-factors in controlling sphingolipid mediated regulation of cellular phenomena.

The backbone of various sphingolipids is generated from the long chain bases sphinganine, sphingosine and in yeast, phytosphinganine. The first, unique and committed reaction to long-chain base synthesis involves the condensation of L-serine with a fatty acid acyl-CoA to generate 3-ketodihydrosphingosine by the enzyme serine palmitoyltransferase (SPT; palmitoyl-CoA; L-serine C-palmityoltransferase (decarboxylating)) (Merrill, 1983). An integral microsomal membrane protein, SPT is composed of at least two subunits, SPT1 and SPT2. The catalytic subunit of SPT is thought to be SPT2 whereas the regulatory activity is thought to be the SPT1 subunit. In yeast, both LCB1 and LCB2 subunits are required for LCB activity and Tsc3p is essential for optimal LCB function.

Since SPT is the key regulatory enzyme in de novo sphingolipid biosynthesis, it is expected that an alteration in SPT activity would affect sphingolipid mediated regulation of cell function. In yeast, SPT has been implicated in heat and hyperosmolar stress responses. Cultured human keratinocytes, when UV irradiated, upregulate SPT activity and show a corresponding increase in SPT2 mRNA and protein levels. SPT activity is increased during apoptosis and governs de novo ceramide synthesis in cells treated with the chemotherapeutic agent, etoposide (Perry, 2000). Inhibition of SPT activity by myriocin, reverses the apoptotic and anti-proliferative effects of a ceramide synthase inhibitor, fumonisin, in pig kidney cells LLCK-1.

Recently, SPT expression has been closely linked to pathophysiological conditions. Procedures such as angioplasty result in vascular injury and in response to this injury, a cascade of events collectively known as restenosis is initiated. An increase in both SPT1 and SPT2 expression has been reported in proliferating vascular smooth muscle cells and fibroblasts in balloon injured rat carotid arteries (D. J. Uhlinger, J. M. Carton, D. C. Argentieri, B. P. Damiano and M. R. D'Andrea, Increased Expression of Serine Pahnitoyltransferase (SPT) in Balloon-injured Rat Carotid Artery, Thromb. Haem., 2001, 86, 1320-6).

Changes in lipid metabolic pathways are among the first events in the de-differentiation of normal cells. Studies have shown that there is a higher percentage of sphingolipids present in the membranes of hepatomas as compared to normal liver cells (Williams R D, Nixon D W, Merrill A H, Jr., Comparison of serine palmitoyltransferase in Morris hepatoma 7777 and rat liver, Cancer Research, 1984, 44(5), 1918-23).

The method of the present invention demonstrates that SPT is expressed abundantly in proliferating fibroblasts in culture and in the hosting “reactive” stromal fibroblasts surrounding the malignant cells in some tumors, which was not observed in the surrounding stromal fibroblasts in normal tissue. Prominent stromal reaction (desmoplasia) is seen in many invasive carcinomas suggesting that stromal cells play a role in cancer pathogenesis (M. Gregoire, B. Lieubeau, The role of fibroblasts in tumor behavior, Cancer Metastasis Rev., 1995, 14(4), 339-50). Reactive fibroblasts, also known as myofibroblasts, are frequently associated with different cancers of epithelial origin and influence the invasive and metastatic potential of carcinoma cells (M. Gregoire, 1995). The present method further demonstrates that the SPT subunits are highly expressed in several established human tumor cell lines and in situ in human malignant cells. In addition, changes in the sub-cellular localization of SPT in proliferating fibroblasts and malignant cells were observed.

The role of nuclear lipid metabolism in signal transduction cascades has recently become apparent. Diacylglycerol kinase, an enzyme involved in phospholipid metabolism, has been shown to localize to the nucleus and is involved in nuclear signal transduction. Sphingosine kinase (SPHK) has also been shown to localize to the nucleus of 3T3 cells within 24 hours of mitogenic stimulation with an increase in nuclear SPHK activity following the treatment (Kleuser B., Maceyka M., Milstien S. and Spiegel S., Stimulation of nuclear sphingosine kinase activity by platelet-derived growth factor, FEBS Lett., 2001, 503(1), 85-90). Sphingolipid metabolites have been shown to be present and active in nuclear preparations lending support to the idea that sphingolipids play a regulatory role in mediating cellular activities from within the nucleus. Our work demonstrates the nuclear association of SPT in proliferating fibroblasts and malignant cells. This represents the second enzyme involved in sphingolipid metabolism that becomes associated with the nucleus upon stimulation. These data suggest a role for sphingolipids as signaling molecules within the nucleus in addition to the previously reported activities in cytoplasmic signaling cascades and as intercellular signaling molecules.

Since SPT activity is altered by a change in the physiological state of the cell, it is imperative to determine the basal levels of this enzyme in normal tissues. The distribution of the SPT1 and SPT2 subunits may serve as a potential marker of cell activity, where high levels of the enzyme may reflect increased metabolic activity (e.g. neutrophil and/or macrophage infiltration, neuronal transmission, exocytosis) or cell proliferation. The SPT1 and SPT2 levels determined in normal tissues and cell types may subsequently be used to analyze cell types in abnormal states. The association of increased SPT expression in pathophysiologic states, such as cancer, inflammation (e.g. ulcerative colitis, inflammatory bowel syndrome, Crohn's Disease, rheumatoid arthritis, atherosclerosis, stroke, asthma) and vascular injury (e.g. restenosis), make it a provocative therapeutic target. Thus, the regulation of SPT may have widespread implications for cellular responses and pathologies because of its prominent position as the enzyme catalyzing the committed and rate-limiting step of the sphingolipid metabolic cascade.

U.S. Pat. No. 6,090,565 describes a method of identifying particular sphingolglycolipid species (specifically a glycoceramide selected from N-tetracosanoyl (lignoceroyl) monoglycosylceramide, N-tetracosanoyl (nervonoyl) monoglycosylceramide, N-docosanoyl monoglycosylceramide and N-linoleoyl monoglycosylceramide) that are indicative of multidrug resistance in certain types of cancer cells (selected from lymphoma, melanoma, sarcoma, leukemia, retinoblastoma, hepatoma, myeloma, glioma, mesothelioma or carcinoma), and the reduction thereof which results in enhanced anticancer agent chemosensitivity. The method of identification includes chromatography; contacting a cancer cell with an antibody or an antibody-component mixture that binds immunologically to an epitope of the sphingolglycolipid species; or, contacting a cancer cell with a purified antibody that is immunologically reactive with a glycoceramide.

U.S. Pat. No. 6,190,894 describes a method and formulation for enhancing penetration of physiologically active substances for cutaneous or transdermal delivery by disrupting the epithelial barrier function using an epithelial barrier-disrupting amount of at least one agent selected from an inhibitor of ceramide synthesis, inhibitor of glucosylcoceramide synthesis, inhibitor of acylceramide synthesis, inhibitor of sphingomyelin synthesis, inhibitor of fatty acid synthesis, inhibitor of cholesterol synthesis, inhibitor of phospholipid and glycosphingolipid (including glucosylceramide, acylceramide and sphingomyelin) degradation, inhibitor of a degradation enzyme of free fatty acid, ceramide, acylceramide or glucosylceramides and sphingomyelin) or both inhibitors and/or stimulators of metabolic enzymes of free fatty acids, ceramide and cholesterol.

### SUMMARY OF THE INVENTION

In one embodiment, the present invention provides a method for measuring the expression level of serine palmitoyltransferase by mammalian cells comprising:

(a) contacting a serine palmitoyltransferase specific compound with a mammalian cell to form a plurality of compound-serine palmitoyltransferase complexes; and,

(b) measuring the level of serine palmitoyltransferase expressed by the cell by detecting the presence of the complexes.

In a second embodiment, the present invention provides a method for measuring the expression of serine palmitoyltransferase by mammalian cells comprising:

(a) contacting a serine palmitoyltransferase specific compound with a first mammalian cell which basally expresses serine palmitoyltransferase to form a first plurality of compound-serine palmitoyltransferase complexes;

(b) contacting a serine palmitoyltransferase specific compound with a second mammalian cell which hyperproliferatively expresses serine palmitoyltransferase to form a second plurality of compound-serine palmitoyltransferase complexes;

(c) determining the levels of serine palmitoyltransferase expressed by the first and second cells by detecting the presence of the first and second plurality of complexes; and

(d) measuring the difference in the levels of serine palmitoyltransferase expressed by the first and second cells.

The measured difference in the levels of serine palmitoyltransferase expressed by the first and second cells is used, for example, to detect or diagnose a cancer, diagnose the metastatic potential of a cancer, monitor the prognosis and progression of a cancer, or monitor the therapeutic efficacy of a treatment of a cancer. The cancer includes, but is not limited to, breast carcinoma, colonic carcinoma, carcinoid, gastric carcinoma, glioma, hepatoma, leiomyosarcoma, liver carcinoma, lung carcinoma, lymphoma, melanoma, mesothelioma, myeloma, ovarian carcinoma, pancreas carcinoma, prostate carcinoma, thyroid carcinoma, renal cell carcinoma, retinoblastoma, rhabdomyosarcoma, sarcoma, undifferentiated carcinoma, and leukemia.

The measured difference in the levels of serine palmitoyltransferase expressed by the first and second cells may also be used to detect or diagnose the occurrence of vascular injury including, but not limited to, restenosis.

The measured difference in the levels of serine palmitoyltransferase expressed by the first and second cells is still further used to detect or diagnose the occurrence of inflammation including, but not limited to, inflammation that results from ulcerative colitis, inflammatory bowel syndrome, Crohn's Disease, rheumatoid arthritis, atherosclerosis, stroke, or asthma.

In another embodiment, the present method provides a method for in vivo imaging of a tissue comprising:

(a) administering to a mammal a serine palmitoyltransferase specific compound, wherein the compound comprises a detectable label, and wherein the compound binds to mammalian cells which hyperproliferatively express serine palmitoyltransferase; and,

(b) determining the location of the cells within a tissue of the mammal by imaging the detectable label.

In yet another embodiment, the present invention provides a method for screening a therapeutically effective compound that inhibits serine palmitoyltransferase comprising:

(a) contacting a serine palmitoyltransferase specific compound with a first mammalian cell which hyperproliferatively expresses serine palmitoyltransferase to form a first plurality of compound-serine palmitoyltransferase complexes;

(b) contacting a potential serine palmitoyltransferase inhibitor compound with a second mammalian cell which hyperproliferatively expresses serine palmitoyltransferase to form a second plurality of compound-serine palmitoyltransferase complexes;

(c) determining the levels of serine palmitoyltransferase expressed by the first and second cells by detecting the presence of the first and second plurality of compound-serine palmitoyltransferase complexes; and,

(d) measuring the difference in the levels of serine palmitoyltransferase expressed by the first and second cells to determine whether the potential inhibitor compound inhibits serine palmitoyltransferase expression.

The serine palmitoyltransferase specific compounds encompassed by the invention include, but are not limited to, a compound that binds to serine palmitoyltransferase, a monospecific antibody that binds to serine palmitoyltransferase or a nucleic acid that will hybridize with serine palmitoyltransferase mRNA.

The mammalian cells which express serine palmitoyltransferase include, but are not limited to, adrenal cells, brain cells, breast cells, colon cells, epithelial cells, endothelial cells, heart cells, immunological cells, kidney cells, liver cells, lung cells, ovary cells, pancreas cells, prostate cells, skin cells, spleen cells, stomach cells, testis cells, thyroid cells, uterus cells or vascular cells. Preferred epithelial cells which express serine palmitoyltransferase include, but are not limited to, endothelial cells, non-glial neuronal cells, colon cells, breast cells, the proximal tubules of the kidney, smooth muscle of the prostate, smooth muscle of the uterus or smooth muscle of the testis. Preferred immunological cells which express serine palmitoyltransferase include polymorphonuclear leukocytes (PMNs), monocytes, macrophages, epitheloid cells, giant cells, microglia, Kupffer cells or alveolar macrophages.

Mammalian cells which basally express serine palmitoyltransferase include, but are not limited to, adrenal cells (cortex, medulla (chromaffin)), brain cells (neuron, astrocyte, oligodendrite or Purkinje), breast cells (epithelium), colon cells (epithelium, mucosal macrophage or smooth muscle), epithelial cells, endothelial cells, heart cells (cardiocyte or endomysium), immunological cells, kidney cells (glomerular endothelial cells or epithelium (distal or proximal tubules)), liver cells (hepatocyte or endothelium), lung cells (epithelium, endothelium or macrophage (dust cells)), ovary cells (epithelium, cortical stroma or myofibroblast), pancreas cells (Islets of Langerhans or Acinar cells), prostate cells (epithelium or smooth muscle), skin cells (epidermis or dermis), spleen cells (sinusoid endothelium, lymphocyte, macrophage or PMNs), stomach cells (epithelium, mucosal macrophage or smooth muscle), testis cells (seminiferous epithelium, Sertoli cells or Leydig cells), thyroid cells (epithelium), uterus cells (epithelium or myometrium) or vascular cells (endothelium or smooth muscle).

Mammalian cells which hyperproliferatively express serine palmitoyltransferase include, but are not limited to, epithelial cells, endothelial cells, cancer cells (selected from breast carcinoma, colonic carcinoma, carcinoid, gastric carcinoma, glioma, hepatoma, leiomyosarcoma, liver carcinoma, lung carcinoma, lymphoma, melanoma, mesothelioma, myeloma, ovarian carcinoma, pancreas carcinoma, prostate carcinoma, thyroid carcinoma, renal cell carcinoma, retinoblastoma, rhabdomyosarcoma, sarcoma, undifferentiated carcinoma or leukemia), immunological cells or cells within a tumor microenvironment.

A still further embodiment of the present method includes a method for treating a serine palmitoyltransferase mediated disorder in a subject in need thereof comprising administering a therapeutically effective amount of a pharmaceutical formulation of a serine palmitoyltransferase inhibitor compound to the subject; wherein, optionally, the serine palmitoyltransferase inhibitor compound is cytotoxic. Preferred serine palmitoyltransferase inhibitor compounds include, but are not limited to, a serine palmitoyltransferase specific compound. In a preferred embodiment of this aspect of the invention, the pharmaceutical formulation is coated onto a balloon catheter or stent and released in a time-dependent manner.

### BRIEF DESCRIPTION OF THE DRAWINGS

FIG. 1 shows the results of an immunoblot analysis to evaluate the specificity of the peptide-specific polyclonal antibodies generated against human SPT1 and SPT2.

FIG. 2 shows SPT expression in normal human brain tissue at 600× magnification.

FIG. 3 shows SPT expression in normal human colon tissue at 600× magnification.

FIG. 4 shows SPT expression in normal human adrenal tissue at 600× magnification.

FIG. 5 shows SPT expression in normal human kidney tissue at 600× magnification.

FIG. 6 shows SPT expression in normal human uterus tissue at 600× magnification.

FIG. 7 shows co-expression of SPT1 and SPT2, respectively, with topoisomerase in normal human colon tissue at 600× magnification.

FIG. 8 shows SPT1 and SPT2 expression in subconfluent fibroblasts at 300× magnification (FIGS. 8A and 8B) and at 600× magnification (FIGS. 8C and 8D).

FIG. 9 shows SPT1 and SPT2 expression in quiescent and wounded fibroblasts at 300× magnification (FIGS. 9A to 9F) and at 600× magnification (FIGS. 9G to 9L).

FIG. 10 shows double staining (IF:IF) of fibroblasts at 1500× magnification.

FIG. 11 shows SPT expression in human tumor cell lines at 900× magnification.

FIG. 12 shows SPT expression in human malignant colonic carcinoma tissue, undifferentiated carcinoma tissue, thyroid carcinoma tissue and sarcoma tissue at 600× magnification.

FIG. 13 shows the characterization of vascular injury in a rat balloon angioplasty model over 14 days.

FIG. 14 shows the time course of the vascular injury response at 1 day, 3 days, 7 days and 3 months after angioplasty.

FIG. 15 shows the dedifferentiation and proliferation of myofibroblasts in the adventitia and matrix remodeling in response to the angioplasty.

FIG. 16 shows activated macrophage and neutrophil infiltration into inflamed colon.

FIG. 17 shows the de novo sphingolipid synthesis pathway.

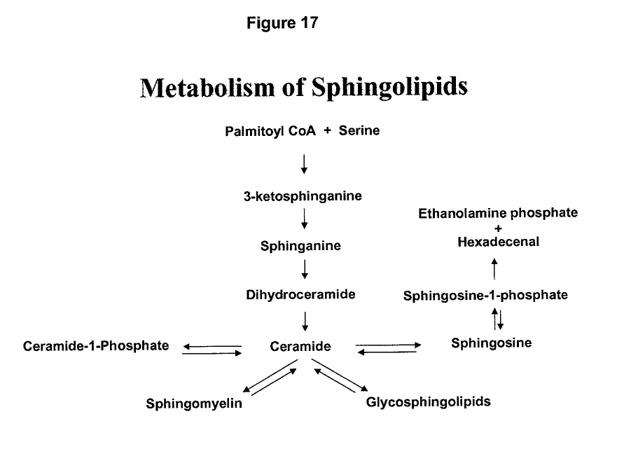

### DETAILED DESCRIPTION OF THE INVENTION

As noted above, mammalian cells which basally express serine palmitoyltransferase include, but are not limited to, adrenal cells (medulla (chromaffin)), brain cells (neuron or Purkinje), breast cells (epithelium), colon cells (epithelium, mucosal macrophage or smooth muscle), epithelial cells, endothelial cells, heart cells (endomysium), immunological cells, kidney cells (glomerular endothelial cells or epithelium (proximal tubules)), liver cells (endothelium), lung cells (epithelium, endothelium or macrophage (dust cells)), ovary cells (cortical stroma or myofibroblast), pancreas cells (Acinar cells), prostate cells (epithelium or smooth muscle), skin cells (epidermis), spleen cells (sinusoid endothelium, macrophage or PMNs), stomach cells (epithelium, mucosal macrophage or smooth muscle), testis cells (seminiferous epithelium, Sertoli cells or Leydig cells), thyroid cells (epithelium), uterus cells (epithelium or myometrium) or vascular cells (endothelium or smooth muscle).

Preferably, mammalian cells which basally express serine palmitoyltransferase include adrenal cells (medulla (chromaffin)), brain cells (neuron or Purkinje), breast cells (epithelium), colon cells (epithelium, mucosal macrophage or smooth muscle), epithelial cells, endothelial cells, immunological cells, kidney cells (glomerular endothelial cells or epithelium (proximal tubules)), liver cells (endothelium), lung cells (epithelium, endothelium or macrophage (dust cells)), ovary cells (cortical stroma or myofibroblast), pancreas cells (Acinar cells), prostate cells (epithelium or smooth muscle), spleen cells (sinusoid endothelium, macrophage or PMNs), stomach cells (epithelium, mucosal macrophage or smooth muscle), testis cells (Leydig cells), thyroid cells (epithelium), uterus cells (epithelium or myometrium) or vascular cells (endothelium or smooth muscle).

More preferably, the mammalian cells which basally express serine palmitoyltransferase include adrenal cells (medulla (chromaffin)), brain cells (neuron), colon cells (mucosal macrophage), epithelial cells, endothelial cells, immunological cells, kidney cells (epithelium (proximal tubules)), lung cells (macrophage (dust cells)), ovary cells (cortical stroma), spleen cells (sinusoid endothelium) or stomach cells (epithelium or mucosal macrophage).

As also noted above, mammalian cells which hyperproliferatively express serine palmitoyltransferase include, but are not limited to, epithelial cells, endothelial cells, cancer cells (selected from breast carcinoma, colonic carcinoma, carcinoid, gastric carcinoma, leiomyosarcoma, ovarian carcinoma, pancreas carcinoma, prostate carcinoma, thyroid carcinoma, renal cell carcinoma, sarcoma, undifferentiated carcinoma or leukemia), immunological cells or cells within a tumor microenvironment.

Preferably, the mammalian cells which hyperproliferatively express serine palmitoyltransferase include epithelial cells, endothelial cells, cancer cells (selected from breast carcinoma, colonic carcinoma, gastric carcinoma, leiomyosarcoma, ovarian carcinoma, pancreas carcinoma, prostate carcinoma, thyroid carcinoma, renal cell carcinoma, sarcoma, undifferentiated carcinoma or leukemia), immunological cells or cells within a tumor microenvironment.

More preferably, the mammalian cells which hyperproliferatively express serine palmitoyltransferase include epithelial cells, endothelial cells, cancer cells (selected from breast carcinoma, colonic carcinoma, leiomyosarcoma, ovarian carcinoma, pancreas carcinoma, prostate carcinoma, thyroid carcinoma, sarcoma or undifferentiated carcinoma), immunological cells or cells within a tumor microenvironment.

Particularly preferred epithelial cells which hyperproliferatively express serine palmitoyltransferase include endothelial cells, non-glial neuronal cells, colon cells, breast cells, the proximal tubules of the kidney, smooth muscle of the prostate, smooth muscle of the uterus or smooth muscle of the testis.

With respect to immunological cells which hyperproliferatively express serine palmitoyltransferase, preferred cells include PMNs, monocytes, macrophages, epitheloid cells, giant cells, microglia, Kupffer cells or alveolar macrophages.

Preferred cells within a tumor microenvironment which hyperproliferatively express serine palmitoyltransferase include stromal fibroblasts, stromal monocytes or myofibroblasts.

As mentioned above, preferred serine palmitoyltransferase specific compounds include a monospecific antibody optionally labeled with a cytotoxic agent or a nucleic acid that will optionally hybridize to serine palmitoyltransferase mRNA.

The present invention provides, for the first time, evidence that the enzyme serine palmitoyltransferase is upregulated in certain tissue disease states. In particular, the two protein subunits that compose the SPT enzyme, SPT1 (a polypeptide having accession number NP006406) and SPT2 (alternatively known as LCB2, a polypeptide having accession number NP004854), are upregulated in disease states where cellular hyperproliferation occurs or in cells with unregulated overexpression of SPT 1 or SPT2 (thus further enabling cellular hyperproliferation).

Therefore, detecting the presence of and measuring the amount of SPT1 or SPT2 in a cell or detecting the presence of SPT1 or SPT2 in vivo provides a method for diagnosing or monitoring disease states, including, but not by way of limitation, cancer and tumor metastasis, inflammation or vascular injury (as in restenosis). Accordingly, inhibiting the upregulation or unregulated overexpression of SPT1 or SPT2 provides a method for treating a disease state mediated by the expression of SPT 1 or SPT2.

Many of the cancer related disease states in question are characterized by overexpression of serine palmitoyltransferase, as in hyperproliferative epithelial cells, hyperproliferative mesenchymal cells, certain immunological cells or cells of the tumor microenvironment.

Particular hyperproliferative epithelial cells where SPT1 and/or SPT2 are expressed in higher amounts than in normal cells include, but are not limited to, cells of the endothelium, non-glial neuronal cells, colon cells, breast cells, the proximal tubules of the kidney or the smooth muscle of the prostate, uterus or testis.

Particular hyperproliferative mesenchymal cells where SPT1 and/or SPT2 are expressed in higher amounts than in normal cells include, but are not limited to, sarcoma cancer cells.

Particular immunological cells that express elevated amounts of serine palmitoyltransferase include, but are not limited to, PMNs, monocytes, macrophages, and specialized macrophages such as epitheloid cells, giant cells, microglia, Kupffer cells or alveolar macrophages.

Other cells located within close proximity to a metastatic tumor microenvironment that hyperproliferatively express serine palmitoyltransferase include, but are not limited to, stromal fibroblasts, stromal monocytes or myofibroblasts.

Other hyperproliferative epithelial, meschymal or immunological cells hyperproliferatively expressing SPT1 and/or SPT2 can be detected using the methods described herein or by other methods well known in the art.

The term “cell” refers to at least one cell, but includes a plurality of cells or fractions of cells, appropriate for the sensitivity of the detection method. Cells suitable for the present invention may be present as isolated, purified cell populations or as a fraction of an organized tissue biopsy. Fractions of a cell may be isolated, for example in a tissue section of a biopsy.

The term “upregulated” or “hyperproliferatively expressed” as used herein means that a greater quantity of the gene product of SPT1 or SPT2 can be detected in the target tissue as compared to a reference sample. A “reference sample” as used herein refers to a sample that demonstrates no detectable disease, and may include, for example, preserved tissue sections from a tissue archive. In particular a reference sample may be an archived sample where the amount of SPT is used to determine the progression of a disease-state. A sample can be an individual cell or cellular fragment containing serine palmitoyltransferase, or the sample may be a component in a larger composition, for example in a tissue section of a biopsy, where the cells of interest may belong to one or more cellular subtypes amongst a field of different cell types.

The phrase “serine palmitoyltransferase specific compound” refers to, for example, synthetic or natural amino acid polypeptides, proteins, small synthetic organic molecules, or deoxy- or ribo-nucleic acid sequences that bind to serine palmitoyltransferase with about 20-fold or greater affinity compared to other proteins or nucleic acids. For example, but not by way of limitation, polyclonal or monoclonal (including classical or phage display) antibodies raised against the serine palmitoyltransferase protein or a peptide fragment thereof or nucleic acid probes that hybridize with serine palmitoyltransferase mRNA are suitable for use in the present invention.

For protein measuring and in vivo imaging embodiments of the present invention, compounds may be labeled compounds with means of direct detection or detection by indirect means, for example by a second labeled compound. For methods directed to treating an SPT mediated disease, the SPT specific compound may be an inhibitor, an antisense nucleotide or a compound labeled with a cytotoxic agent. Small molecule inhibitors are known and generally are based on structural homology to serine or sphingosine.

Examples of serine palmitoyltransferase inhibitor compounds include, but are not limited to, myriocin (CAS registry number 35891-70-4), 3-chloro-D-alanine (CAS registry number 39217-38-4), L-cycloserine (CAS registry number 339-72-0), and D-serine (312-84-5). Novel inhibitors are discovered using methods that measure serine palmitoyltransferase enzymatic activity. Compounds that are labeled with a cytotoxic agent include, for example, an antibody that is labeled with a cytotoxic agent and that immunologically reacts with serine palmitoyltransferase. Methods to label antibodies with cytotoxic agents are well known in the art.

The phrase “labeled compound” refers to moieties capable of measurement comprising radioactive atoms, enzymes, fluorescent molecules, or alternative tags, for example biotin. Proteins, peptides, carbohydrates, and nucleic acids are conjugated to a detectable label using techniques well known in the art and described, for example, in Bioconjugate Techniques, by G. T. Hermanson, Academic Press publishers, 1996. Particular radioisotopes useful as a label in the present invention are ³H, ¹²⁵I, ¹³¹I, ³⁵S, ³²P, ³³P, ²¹²⁻Bi, ⁹⁰Y, ⁸⁸Y, ⁹⁹Tc, ⁶⁷Cu, ¹⁸⁸Re, ⁶⁶Ga, ⁶⁷Ga, ¹¹¹In, ^{114m}In, ¹¹⁵In, or ¹⁰B and others that are known in the art. Radioisotopes are introduced into a polypeptide by conventional means, known to those skilled in the art, such as iodination of a tyrosine residue, phosphorylation of a serine or threonine residue, or incorporation of tritium, carbon or sulfur utilizing radioactive amino acid precursors. Other radioactive atoms are introduced using bifunctional chelating agents that cross-link a metal chelating moeity onto a polypeptide. Particular examples of enzymes suitable for use in the present invention are horseradish peroxidase, alkaline phosphatase, or luciferase. A preferred example of a detectable label is an enzyme that cleaves a substrate to yield a chromogenic or luminescent product. Particular examples of fluorescent molecules useful in the methods of the present invention include, but are not limited to, coumarins, xanthene dyes such as fluoresceines, rhodols, and rhodamines, resorufins, cyanine dyes bimanes, acridines, isoindols, dansyl dyes, aminophthalic hydrazides such as luminol and isoluminol derivatices, aminophthalimides, aminonapthalimides, aminobenzofurans, aminoquinolines, dicanohydroquinones, and europium and terbium complexes and related compounds. Direct measurement is conducted by observing the presence of the radioactive atom or flourogenic molecule, or by observation of enzymatic activity of a colorimetric or luminescent substrate. Indirect measurement is conducted by adding an additional compound including a label to the test sample so that it can interact with the compound bound to the test sample. A well-known example is when the labeled compound comprises biotin, and a second compound comprises avidin or streptavidin and a detectable label. A second well-known example is when a first antibody is used to bind to the serine palmitoyltransferase protein and is detected with a second anti-antibody comprising a detectable label.

One embodiment of the present invention relates to methods for measuring SPT1 or SPT2 hyperproliferatively expressed in a cell comprising contacting the cell with a serine palmitoyltransferase specific compound and measuring or detecting the formation of a serine palmitoyltransferase-compound complex as a result of compound binding to the SPT. The method to detect serine palmitoyltransferase can be further defined by comparing changes in the amount of serine palmitoyltransferase in the cell with a reference sample. Advantageously, using the methods described herein, expression of SPT1 or SPT2 can be used to determine if a tissue contains cell which have hyperproliferative expression of SPT1 or SPT2 in the cells. In one embodiment, the methods of the present invention are used to diagnose a cancer or a metastatic tumor, monitor the prognosis and progression of tumor metastasis or monitor the therapeutic efficacy of any intervention or treatment of a cancer or a metastatic tumor. In other embodiments, the methods of the present invention are used to diagnose the occurrence of vascular injury (as in restenosis) or inflammation or monitor the therapeutic efficacy of any intervention or treatment of restenosis or inflammation.

The novel methods described in the present invention describe how the upregulation of SPT1 or SPT2 protein can be visualized in various pathophysiologic states. The methods of visualizing such upregulation may also be applied to SPT1 or SPT2 nucleic acid and can be performed using methods well known in the art including, but not limited to, hybridization techniques with a labeled nucleic acid probe or by quantitative RT-PCR. Other methods of visualizing SPT1 or SPT2 protein can be performed using methods well known in the art including, but not limited to, affinity detection methods, Western blotting, fluorescent flow cytometry methods or immunohistological/immunocytological methods.

In these techniques, generally, the protein of interest is labeled with a specific probe and detected via the degree probe incorporation to the sample. In flow cytometry, the cells are analyzed in a solution, whereas in cellular imaging techniques, a field of cells is compared for the amount of probe binding. In a generally preferred embodiment, an antibody is used as a probe and is labeled with a detectable probe such as a radioactive atom or a fluorescent molecule.

Another embodiment of the present invention relates to detection of SPT by in vivo imaging. Accordingly, a labeled serine palmitoyltransferase specific compound is administered to a mammal, the labeled compound binds to SPT1 or SPT2 and the location of the labeled compound is measured as a method to image the location of a hyperproliferative cell.

In a particular embodiment of the present invention, an antibody is labeled with a radioactive atom and is used to measure the presence of the SPT1 or SPT2 protein in vivo, as is well known in the art. Using this method, presence and location of a metastatic tumor can be the imaged visually by autoradiological techniques or by an auditory signal using a device that converts photon emissions to an audible report, as described in U.S. Pat. No. 4,782,840 to Martin et al. In another embodiment, a probe (in particular, an antibody) can be labeled with a chromophore that absorbs light in the range of about 300nm to about 1300 nm, such that the SPT1 or SPT2 can be imaged using fluorescence detection. Classes of chromophores that absorb light in the range of 300-1300 nm are described in PCT application PCT/GB98/02833, to Towler et al.

Another aspect of the present invention relates to treating a disease state mediated by the presence of unregulated SPT in a hyperproliferative cell. The methods of treating disease comprise inhibition of SPT enzymatic activity, reduction in the amount of SPT expression within the cell or contact of the hyperproliferative cell with a cytotoxic serine palmitoyltransferase compound. In one embodiment, the inhibitor is a small molecule inhibitor of serine palmitoyltransferase enzymatic activity.

In another embodiment of the present invention, methods to limit or prevent the progression of a metastatic tumor comprise administration of a compound that reduces the expression of serine palmitoyltransferase. In a particular embodiment, the expression of serine palmitoyltransferase is reduced using an antisense nucleic acid that will hybridize to either the SPT1 or the SPT2 mRNA.

In another embodiment, the hyperproliferative cell is treated with a cytotoxic, labeled serine palmitoyltransferase specific compound. In a particular embodiment, the cytotoxic serine palmitoyltransferase specific compound is used to limit or prevent the progression of a metastatic tumor.

Advantageously a serine palmitoyltransferase inhibitor is administered to a subject with a malignancy with at least one other non-platinum and platinum containing anti-tumor agent. For example, but not to limit the present invention, an anti-serine palmitoyltransferase compound can be administered in a dosing regimen with a cytotoxic compound, such as a DNA alkylating agent, or with an anti-angiogenic compound. Preferred anti-tumor agents are selected from the group consisting of cladribine (2-chloro-2′-deoxy-(beta)-D-adenosine), Chlorambucil (4-[bis(2-chlorethyl)amino]benzenebutanoic acid), DTIC-Dome (5-(3,3-dimethyl-1-triazeno)-imidazole-4-carboxamide), platinum chemotherapeutics and nonplatinum chemotherapeutics. Platinum containing anti-tumor agents include, but are not limited to, cisplatin (cis-dichlorodiamineplatinum). Non-platinum containing anti-tumor agents include, but are not limited to, cyclophosphamide, fluorouracil, epirubicin, methotrexate, vincristine, doxorubicin, bleomycin, and etoposide. Each anti-tumor agent is administered within therapeutically effective amounts, which are well known in the art, and vary based on the agent used, the type of malignancy, and other conditions.

In another embodiment, an inhibitor of serine palmitoyltransferase is coated onto a balloon-catheter or stent such that it is released in a site-directed and time dependent manner. Such devices are useful to prevent the occurrence of restenosis by inhibiting serine palmitoyltransferase and thus preventing hyperproliferation of the endothelium. The methods of treating an SPT mediated disease include use of small molecule therapeutic agents or inhibitor compounds delivered or “seeded” directly or indirectly into tissues with disease states wherein SPT expression is upregulated, where cellular hyperproliferation occurs or in cells with unregulated overexpression of SPT.

The instant pharmaceutical compositions are prepared according to conventional pharmaceutical techniques. A pharmaceutically acceptable carrier may be used in the composition of the invention. The composition may take a wide variety of forms depending on the form of preparation desired for administration including, but not limited to, intravenous (both bolus and infusion), oral, nasal, transdermally, topical with or without occlusion, intraperitoneal, subcutaneous, intramuscular, or parenteral, all using forms well known to those of ordinary skill in the pharmaceutical arts. In preparing the compositions in oral dosage form, one or more of the usual pharmaceutical carriers may be employed, such as water, glycols, oils, alcohols, flavoring agents, preservatives, coloring agents, syrup and the like in the case of oral liquid preparations (for example, suspensions, elixirs and solutions), or carriers such as starches, sugars, diluents, granulating agents, lubricants, binders, disintegrating agents and the like in the case of oral solid preparations (for example, powders, capsules and tablets).

The serine palmitoyltransferase inhibitory compounds may alternatively be administered parenterally via injection of a formulation consisting of the active ingredient dissolved in an inert liquid carrier. The injectable formulation can include the active ingredient mixed with an appropriate inert liquid carrier. Acceptable liquid carriers include vegetable oils such as peanut oil, cotton seed oil, sesame oil, and the like, as well as organic solvents such as solketal, glycerol formal, and the like. As an alternative, aqueous parenteral formulations may also be used. For example, acceptable aqueous solvents include water, Ringer's solution and an isotonic aqueous saline solution. Further, a sterile non-volatile oil can usually be employed as solvent or suspending agent in the aqueous formulation. The formulations are prepared by dissolving or suspending the active ingredient in the liquid carrier such that the final formulation contains from 0.005 to 10% by weight of the active ingredient. Other additives including a preservative, an isotonizer, a solubilizer, a stabilizer and a pain-soothing agent may adequately be employed.

A compound used in the methods of the present invention can also be administered in the form of liposome delivery systems, such as small unilamellar vesicles, large unilamellar vesicles and multilamellar vesicles. Liposomes containing delivery systems as well known in the art are formed from a variety of phospholipids, such as cholesterol, stearylamine or phosphatidylcholines.

As used herein, a “therapeutically effective amount” of the instant pharmaceutical composition, or compound therein, means an amount that inhibits the function of the serine palmitoyltransferase activity. The instant pharmaceutical composition will generally contain a per dosage unit (e.g., tablet, capsule, powder, injection, teaspoonful and the like) from about 0.001 to about 100 mg/kg. In one embodiment, the instant pharmaceutical composition contains a per dosage unit of from about 0.01 to about 50 mg/kg of compound, and preferably from about 0.05 to about 20 mg/kg. Methods are known in the art for determining therapeutically effective doses for the instant pharmaceutical composition. The effective dose for administering the pharmaceutical composition to a human, for example, can be determined mathematically from the results of animal studies. Furthermore, compounds of the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

### BIOLOGICAL EXAMPLES

The following examples illustrate the present invention without, however, limiting the same thereto.

### Example 1

Cell Identification Antibodies

Antibody Production

Rabbit polyclonal antibodies to the SPT subunits were generated using antigenic peptide sequences predicted by the algorithm of Hopp/Woods. The peptides utilized for antibody production against the human SPT1 subunit (accession number Y08685) were: SEQ.ID.NO.:1 (KLQERSDLTVKEKEEC, corresponding to residues 45-59); and SEQ.ID.NO.:2 (KEQEIEDQKNPRKARC, corresponding to residues 222-236). The peptides used as antigens for the human SPT2 subunit (accession number Y08686) were: SEQ.ID.NO.:3 (CGKYSRHRLVPLLDRPF, corresponding to residues 538-552); and SEQ.ID.NO.:4 (CGDRPFDETTYEETED, corresponding to residues 549-561).

A cysteine and glycine were added to the amino terminus of these peptides to allow for KLH conjugation and decreased steric hindrance for the coupling. Rabbit polyclonal antibodies were raised against both peptides separately for each SPT subunit. The resulting immune sera were pooled and the mixed polyclonal antisera was used as the source of antibody against the specific SPT subunit. The IgG fractions were used at 2 μg/mL.

Antibody Characterization

The specificity of the rabbit anti-SPT1 or rabbit anti-SPT2 polyclonal antibodies was evaluated by immunoblot analysis. Microsomal membranes from HEK 293 cells were prepared. Fifty micrograms of microsomal membrane protein were fractionated in each of four lanes of a SDS-polyacrylamide gel. After transfer to a nitrocellulose membrane, the membrane was probed with a 1:1000 dilution of peptide specific polyclonal antibodies prepared as described above. Bound antibody was detected with an alkaline phosphatase conjugated goat anti-rabbit IgG (Santa Cruz).

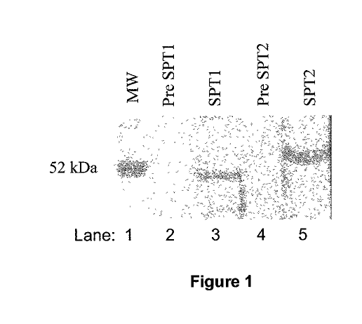

FIG. 1 shows that the antibodies recognized proteins of the predicted molecular weights for SPT1 and SPT2 in HEK293 microsomal membrane preparations and did not react with other proteins contained within the preparation.

The primary monoclonal and polyclonal antibodies to vimentin shown in Table 1 were utilized in normal human tissues to demonstrate tissue antigenicity and reagent quality. The negative controls included replacement of the primary antibody with the same species IgG isotype non-immunized serum.

TABLE 1

| Name | Type | Titer | Vendor |
|---|---|---|---|
| Non-immunized serum | Polyclonal, IgG | 2.0 μg/ml | Vector Labs, CA |
| Non-immunized serum | Monoclonal, IgM | 2.5 μg/ml | Vector Labs, CA |
| Pre-immune serum | Polyclonal, IgG | 2.0 μg/ml | RWJPRI, NJ |
| SPT1 | Polyclonal, IgG | 2.0 μg/ml | RWJPRI, NJ |
| SPT2 | Polyclonal, IgG | 2.0 μg/ml | RWJPRI, NJ |
| Smooth muscle actin | Monoclonal, IgM | 2.0 μg/ml | Dako, CA |
| Vimentin | Monoclonal, IgM | 2.0 μg/ml | Dako, CA |

The primary monoclonal and polyclonal antibodies to vimentin shown in Table 2 were utilized in hyperproliferative human tissues to demonstrate tissue antigenicity and reagent quality. The negative controls included replacement of the primary antibody with the same species IgG isotype non-immunized serum. In addition, the antibodies were pre-absorbed with their specific antigen overnight in a 10-fold titer excess of antigen as another negative control.

TABLE 2

| Name | Type | Titer | Vendor |
|---|---|---|---|
| Topoisomerase IIα | Monoclonal, IgM | 1.0 μg/mL | Pharmingen, CA |
| Macrophage (CD68) | Monoclonal, IgM | 1.0 μg/mL | Dako, CA |
| Preimmunized serum | Polyclonal, IgG | 2.0 μg/mL | RWJPRI, NJ |
| (SPT-1) | | | |
| Preimmunized serum | Monoclonal, IgM | 1.0 μg/mL | RWJPRI, NJ |
| (SPT-2) | | | |
| SPT-1 | Polyclonal, IgG | 2.0 μg/mL | RWJPRI, NJ |
| SPT-2 | Polyclonal, IgG | 1.0 μg/mL | RWJPRI, NJ |
| Smooth muscle actin | Monoclonal, IgM | 2.0 μg/mL | Dako, CA |
| Vimentin | Monoclonal, IgM | 2.0 μg/mL | Dako, CA |

### Example 2

Immunohistochemistry (IHC) of Normal Human Tissues

Commercial human normal and tumor checkerboard tissue slides (Dako, Carpenteria, Calif.; Biomeda, Foster City, Calif.) were deparaffinized, hydrated and processed for routine IHC. Briefly, slides were microwaved in Target (Dako), cooled, placed in phosphate-buffered saline (pH 7.4, PBS) then placed in 3.0% H₂O₂. Slides were processed through an avidin-biotin blocking system according to the manufacturer's instructions (Vector Labs, Burlingame, Calif.) and then placed in PBS. All reagent incubations and washes were performed at room temperature. Normal blocking serum (Vector Labs) was placed on all slides for 10 min. After brief rinsing in PBS, primary antibodies (Table 1) were placed on slides for 30 min. The slides were washed and biotinylated secondary antibodies, goat anti-rabbit (polyclonal antibodies) or horse anti-mouse (monoclonal antibodies) were placed on the tissue sections for 30 min (Vector Labs). After rinsing in PBS, the avidin-horse-radish peroxidase-biotin complex reagent (ABC, Vector Labs) was added for 30 min. Slides were washed and treated with the chromogen 3,3′-diaminobenzidine (DAB, Biomeda), rinsed in dH₂O, and counterstained with hematoxylin.

Double Immunohistochemisty Labeling (IHC:IHC)

Protocols for simultaneous double immunohistochemical labeling (IHC:IHC) have been previously published and are similar to those cited for single immunohistochemical labeling except the slides were not processed for counterstaining after the second chromogen step of the first antigen detection protocol. Instead, the slides were placed into PBS.

The second antigen was detected by an alkaline phosphatase-Fast Red (Vector Labs, CA; Dako, Calif.) system. The primary antibody was placed on the slides for 30 min at room temperature. After brief washing, the secondary biotinylated antibody was added for 30 min at room temperature. The slides were then washed in PBS and then the streptavidin-alkaline phosphatase (Vector Labs, CA) reagent was placed on the slides for 30 minutes at room temperature. After washing, the Fast Red chromogen (Dako, Calif.) was placed on the slides for 2 times 5 minutes. Subsequently, the slides were processed for routine counterstaining in hematoxylin, washed and then coverslipped in a water based mounting media (Dako, CA) for viewing under a BX-50 Olympus light microscope.

Multiple controls were performed to insure proper interpretations of the labeling on the slides. The primary antibodies were substituted with the proper species isotype to control for the detection systems. On another set of controls, the first primary was omitted and the second primary antibody was processed, and vice versa.

Specificity of SPT1 and SPT2 Antibodies

Rabbit polyclonal antibodies specific for the two human SPT subunits were generated and the specificity of the antibodies was demonstrated in the Immunoblot shown in FIG. 1.

Fifty micrograms of HEK 293 microsomal membrane proteins were fractionated on four lanes of an SDS-polyacrylamide gel. After transferring the proteins to a nitrocellulose membrane, the four lanes were cut apart and probed separately with a 1:1000 dilution of protein G purified antibody from either the SPT 1 pre-immune serum (Pre SPT1), the SPT1 anti-serum (SPT1), SPT2 pre-immune serum (Pre SPT2), or the SPT2 anti-serum (SPT2). Bound antibody was detected using a 1:2000 dilution of alkaline phosphatase conjugated goat anti-rabbit IgG. FIG. 1 shows a band of the expected molecular weight for SPT1 (55 kD) and SPT2 (65 kD).

Microsomal membrane fractions obtained from wild type HEK cells were resolved by SDS-PAGE and the western blot was probed with either pre-immune serum or the SPT-specific polyclonal antibodies. Single immunoreactive bands of the expected molecular weights were observed, specifically, Mr˜55 kDa for SPT1 (lane 3) and Mr˜65 kDa for SPT2 (lane 5); non-specific binding was not observed with the pre-immune serum (lanes 2 and 4)

Tissue Distribution of SPT1 and SPT2

The analysis for SPT1 and SPT2 protein expression in normal human tissues was obtained using IHC and the distribution of SPT1 and SPT2 in human tissues is presented in Table 3. Table 3 does not reflect differences observed between SPT1 and SPT2 immunolabeling.

Formalin-fixed, paraffin embedded tissues were used in a multi-tissue format to eliminate potential staining artifacts such as slide to slide and run to run variability. Table 1 lists the positive and negative controls in addition to the experimental antibodies. Positive labeling was defined by the strength of brown stain and scored according to the following criteria: 1) no immunoreactivity was scored as negative (N); 2) light brown immunoreactivity was scored as weak (W); 3) brown immunoreactivity was scored as moderate (M) and 4) dark brown immunoreactivity was scored as strong (S). The negative controls did not produce observable labeling. The number of labeled cells for SPT 1 and SPT2 in a 100 × viewing field in normal human tissues was n=2−10); Negative (N) had no labeled cells; Weak (W) had 1-10 labeled cells; Moderate (M) had 11-20 labeled cells; Strong (S) had >20 labeled cells.

TABLE 3

| Tissue | Cell Types | SPT1 | SPT2 |
|---|---|---|---|
| Adrenal | Cortex | N | N |
| | Medulla (chromaffin cells) | S | S |
| Brain | Neurons | S | S |
| | Astrocytes | N | N |
| | Oligodendrites | N | N |
| | Purkinje cells | M | M |
| Breast | Epithelium | M | M |
| Colon | Epithelium | M | M |
| | Mucosal macrophages | S | S |
| | Smooth muscle | M | M |
| Heart | Cardiocytes | N | N |
| | Endomysium | W | W |
| Kidney | Glomerular endothelial cells | M | M |
| | Epithelium: distal tubule | N | N |
| | Epithelium: proximal tubule | S | S |
| Liver | Hepatocytes | N | N |
| | Endothelium | M | M |
| Lung | Epithelium | M | M |
| | Endothelium | M | M |
| | Macrophages (dust cells) | S | S |
| Ovary | Epithelium | N | N |
| | Cortical stroma | S | S |
| | Myofibroblasts | M | M |
| Pancreas | Islets of Langerhans | N | N |
| | Acinar cells | M | M |
| Prostate | Epithelium | M | M |
| | Smooth muscle | M | M |
| Skin | Epidermis | W | W |
| | Dermis | N | N |
| Spleen | Sinusoid endothelium | S | S |
| | Lymphocytes | N | N |
| | Macrophages, PMNs | M | M |
| Stomach | Epithelium | S | S |
| | Mucosal macrophages | S | S |
| | Smooth muscle | M | M |
| Testis | Seminiferous epithelium | W | W |
| | Sertoli cells | W | W |
| | Leydig cells | M | M |
| Thyroid | Epithelium | M | M |
| Uterus | Epithelium | M | M |
| | Myometrium | M | M |
| Vascular | Endothelium | M | M |
| | Smooth muscle | M | M |

Generally, the vascular endothelium and smooth muscle cells located throughout the human tissues were moderately immunopositive for SPT1 and SPT2. Except for the ovarian epithelium, the epithelial cells in all tested tissues were moderate to strongly immunopositive for SPT1 and SPT2. In addition, mucosal macrophages from the colon, lung and stomach were strongly immunopositive for SPT1 and SPT2. In the spleen, in addition to the macrophages, the polymorphonuclear cells (PMN) stained positive for both SPT1 and SPT2 but no reactivity was observed in the lymphocytes. The colon, lung, prostate, stomach, thyroid, uterus and vascular tissues were moderate to strongly immunopositive for SPT1 and SPT2. However, SPT1 and SPT2 are either weakly present or completely undetectable in the skin and heart tissues.

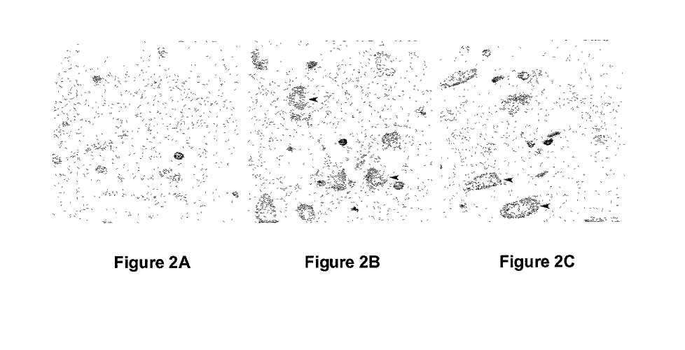

FIGS. 2-7 present some of the normal human tissues tested for SPT1 and SPT2 expression by immunohistochemistry.

FIG. 2 shows normal brains immunolabeled with pre-immune serum (FIG. 2A), SPT1 (FIG. 2B) and SPT2 (FIG. 2C) specific antibodies respectively. In the cerebral cortex, the Pyramidal neurons showed positive intracellular immunoreactivity for SPT1 and SPT2. Both SPT1 and SPT2 were localized in the neuronal cytoplasm and the expression levels of both subunits appeared similar. Purkinje cells found in the human cerebellum were moderately immunopositive for both SPT1 and SPT2 (data not shown). In contrast, SPT1 and SPT2 were not detectable in other neuronal cell types such as the astrocytes, microglia and oligodendritic cells.

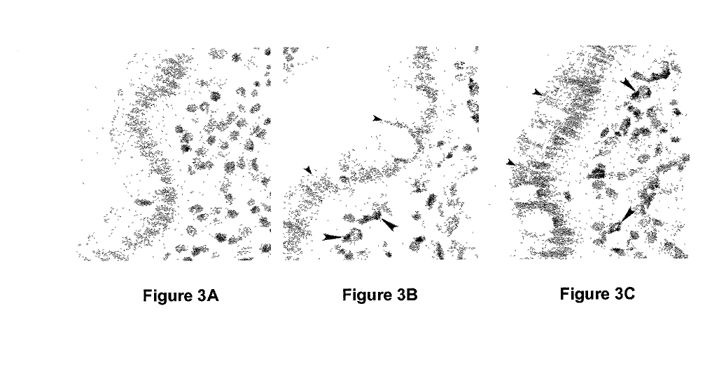

FIG. 3 shows normal human large intestine immunolabeled with pre-immune serum (FIG. 3A), SPT1 (FIG. 3B) and SPT2 (FIG. 3C) antibodies. Epithelial cells (small arrowheads) and macrophages (large arrowheads) show positive intracellular immunoreactivity for SPT1 (FIG. 3B) and SPT2 (FIG. 3C). As in the neurons, expression of both SPT1 and SPT2 was mainly cytoplasmic. In comparison to the moderate expression of SPT2 in the epithelial cells, the mucosal macrophages exhibited a much stronger immunoreactivity to SPT2. No immunoreactivity was observed in any cell type upon staining with the pre-immune serum (3A). The high expression of SPT in mucosal macrophages in the colon (FIGS. 3A and 3B) and stomach and dust cells (alveolar macrophages) (Table 3) may be due to the fact that these macrophages are associated with tissues that are prone to environmental exposure and thus may have been activated.

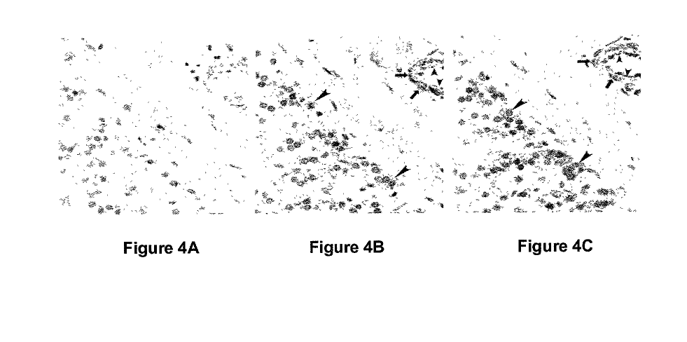

FIG. 4 shows normal human adrenal glands immunolabeled with either pre-immune serum (FIG. 4A) or SPT1 (FIG. 4B) and SPT2 (FIG. 4C) specific antibodies respectively. Chromaffin cells of the adrenal medulla (large arrowheads), vascular smooth muscle cells (arrows) and endothelium (small arrowheads) show strong, positive cytoplasmic immunoreactivity for SPT1 and SPT2. SPT1 and SPT2 expression was undetectable in the adrenal cortex. SPT2 expression in the endothelium and the chromaffin cells was greater than the expression of SPT1. Also, besides the cytoplasm, SPT2 expression can be clearly observed in the chromaffin cell nuclei. No detectable SPT1 or SPT2 immunolabeling is present in the supporting stromal fibroblasts.

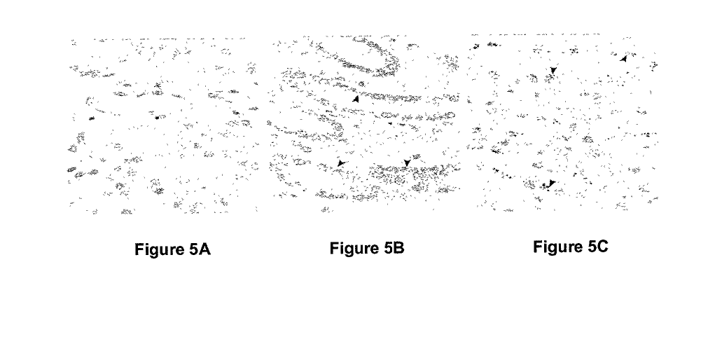

FIG. 5 shows immunolabelling of normal human kidneys with pre-immune serum (FIG. 5A), SPT1 (FIG. 5B) and SPT2 (FIG. 5C) specific antibodies. The proximal tubules (arrowheads) showed positive immunoreactivity for SPT1 and SPT2. SPT1 presented diffuse intracellular labeling patterns in the epithelial cells of the proximal tubules which was different from the punctate labeling pattern of SPT2 in the same cell types. SPT1 expression was diffuse in the cytoplasm whereas SPT2 immunostaining is more punctate and overall weaker than SPT1. No immunoreactivity was observed with the pre-immune serum. FIG. 5C shows SPT activity localized to the cytosolic side of the endoplasmic reticulum, with the punctate appearance of SPT2 expression in the renal proximal tubule epithelium, thus suggesting SPT2 expression in association with the endoplasmic reticulum.

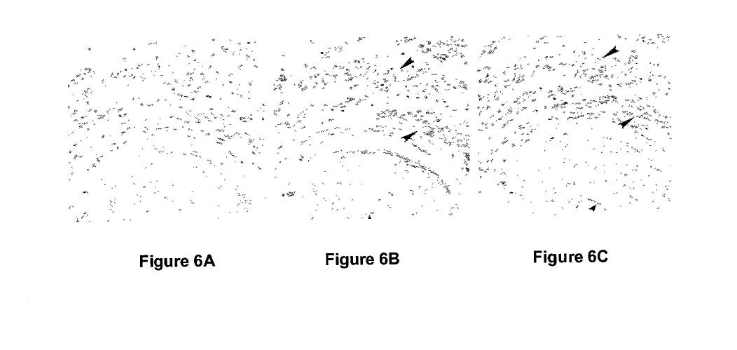

FIG. 6 shows several normal human uteri tissue samples immunolabeled with either pre-immune serum (FIG. 6A) or SPT1 (FIG. 6B) and SPT2 (FIG. 6C) specific antibodies respectively. Uterine stromal smooth muscle cells (large arrowheads) and endothelium (small arrowheads) show positive immunoreactivity for SPT1 and SPT2, with higher SPT2 expression (compared to SPT1) in the endothelial cells. No immunoreactivity was observed with the preimmune serum.

The method of the present invention shows that SPT1 and SPT2 is expressed in metabolically active cells (such as the adrenal chromaffin cells that secrete epinephrine and nor-epinephrine on autonomic nervous stimulation) and in neurons and ovarian cortical stromal cells. Since SPT1 and SPT2 positive labeling was observed in proliferating cell types such as the epithelial layers in the stomach, lungs (data not shown), renal proximal tubules and colonic lumen, double immunohistochemical labeling of the human large intestines was performed.

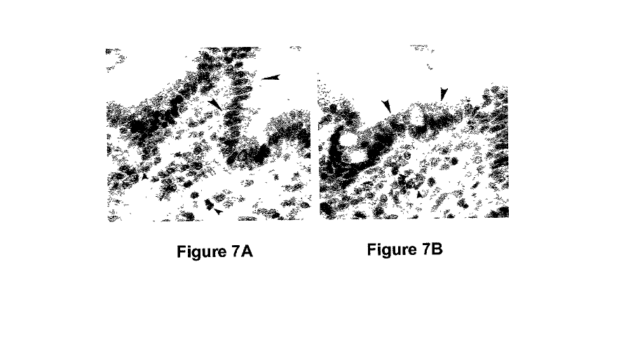

FIG. 7 shows the double immunohistochemical labeling of the human large intestines using antibodies to topoisomerase IIα in red (a marker of cell proliferation and SPT 1) (FIG. 7A) and SPT2 (FIG. 7B) specific antibodies (in brown). Arrowheads show the co-localization of red and brown labeling cells indicating that SPT1 and SPT2 are expressed in proliferating epithelial cells (large arrowheads). Small arrowheads show the presence of SPT1 (FIG. 7A) and SPT2 (FIG. 7B) in macrophages.

Results

The method of the present invention characterizes the distribution of serine palmitoyltransferase subunits SPT1 and SPT2 in normal human tissues and gives insight into the similarities and possible functions of SPT1 and SPT2. The differences observed in expression of SPT1 and SPT2 indicated that the localization and expression levels of SPT may be linked to the physiological state of the cell.

Metabolically active cells and proliferating cells expressed higher levels of SPT. The differences between SPT1 and SPT2 expression in the same cell type within the same tissue suggested that there must be specific, possibly independent functions of each subunit in enabling SPT activity. Unlike yeast, overexpression of murine SPT2 alone in human HEK293 cells results in a corresponding increase in SPT activity, whereas SPT1 alone does not increase SPT activity (Weiss and Stoffel, 1997). In addition to SPT1 and SPT2, human SPT may also have additional components like the Tsc3p protein in yeast. Also, the localization of SPT2 in the nuclei may suggest that SPT2 associates with another nuclear protein(s) or is modified and transported to the nucleus. Thus, analysis of the difference in dynamics of SPT1 and SPT2 expression will help in elucidating SPT activity.

A knowledge of SPT expression in normal cells can be used to measure abnormal cellular activity in proliferative disorders such as cancers. Both, the absolute level of expression of SPT and the localization of enzyme activity may be indicative of an alteration in cell physiology. The increase in SPT activity observed in pathophysiological conditions such as vascular hyperplasia (D. J. Uhlinger, J. M. Carton, D. C. Argentieri, B. P. Damiano and M. R. D'Andrea, Increased Expression of Serine Palmitoyltransferase (SPT) in Balloon-injured Rat Carotid Artery, Thromb. Haem., 2001, 86, 1320-6), wound healing and tumors suggests therapeutic potential for SPT. Inhibiting or lowering SPT activity in these conditions might affect the symptoms associated with the conditions. In porcine epithelial kidney cells, LLC-PK1, fumonisin induced cytotoxity and anti-proliferative effects were reduced on treating the cells with SPT1 specific inhibitors like myriocin. In the same study, intraperitoneal administration of myriocin to BALB/C mice reduced free sphingosine accumulation in the kidney by 60% with no apparent clinical side-effects. Thus, SPT inhibitors such as myriocin may have important therapeutic potential in treatment of proliferative disorders such as cancer and may affect pathophysiologies associated with conditions such as inflammation and vascular injury.

The method of the present invention provides the first direct immunolocalization and comparison of SPT1 and SPT2 expression in normal human tissues and is a critical step towards elucidating the complexity of SPT activity in the cell. Understanding the role of these components in SPT activity is imperative in determining the regulation of the numerous, critical, sphingolipid mediated cellular functions and responses in various diseased states such as cancer and restenosis.

### Example 3

Immunohistochemistry of Hyperproliferative Human Tissues

Using the IHC procedure of Example 2, SPT1 and SPT2 proteins were localized using IHC on formalin-fixed, paraffin embedded tissues. Normal and malignant human tissues were assayed simultaneously in a multi-tissue format to eliminate potential staining artifacts such as slide to slide and run to run variability.

Cell Culture

Human neonatal dermal fibroblasts and their culture media were obtained from Clonetics/BioWhittaker (Walkersville, Md.). Cell suspensions (5×10⁴/ML) were seeded in 4-well chamber slides (NUNC, Naperville, Ill.) for immunocytochemistry. To mimic the in vivo activation of differentiated, quiescent fibroblasts in vitro a scrape-wounding model was used. Briefly, cells were incubated for either 2 days (subconfluent, proliferative conditions), 9 days (hyperconfluent, quiescent conditions) or a 9-day quiescent culture was subjected to mechanical scraping using the end of a pipette. To assess expression in the wound response the cells were cultured for an additional 5 days after wounding.

Immunocytochemistry (ICC)

Four-chambered cultured slides were fixed with 10% neutral buffered formaline for 10 minutes at room temperature, rinsed in PBS and then assayed for ICC. All washing steps were performed using automation buffer with tween-20 (Research Genetics, Huntsville, Ala.).

Double Immunohistochemistry (IHC:IHC)

To determine if SPT was co-localized in proliferating cells, IHC:IHC was used to simultaneously detect SPT 1 or SPT2 expression with detection of a proliferation marker, proliferating cell nuclear antigen (PCNA). Briefly, slides were first processed for single IHC labeling protocols for detection of SPT-1 or SPT-2 as described above. Without processing the slides for hematoxylin, PCNA antibodies (Pharminigen, San Diego, Calif.) were placed on the tissues for 30 min. After several PBS washes, the biotinylated horse anti-mouse secondary antibodies (Vector Labs) were similarly incubated. The presence of PCNA positive cells was visualized using an alkaline phosphatase detection system through incubation with alkaline phosphatase conjugated ABC (Vector Labs) followed by development using the Fast Red chromogen (Sigma). Slides were then routinely counterstained and mounted.

Table 4 represents the immunolocalization of SPT1 and SPT2 in a variety of human malignant tissues. The positive labeling was defined by the presentation of brown staining and was scored according to the number of labeled cells for SPT1 and SPT2 in a 100× viewing field in 18 different human tumors. Five carcinomas (colon, ovarian, pancreas, thyroid, undifferentiated) and two sarcomas demonstrated strong over expression of both SPT subunits. The negative controls did not produce observable labeling. The number of labeled cells for SPT1 and SPT2 in a 100× viewing field in malignant human tissues was n=2−10); Negative (N) had no labeled cells; Weak (W) had 1-10 labeled cells; Moderate (M) had 11-20 labeled cells; Strong (S) had >20 labeled cells.

TABLE 4

| Tissue | SPT-1 | SPT-2 |
|---|---|---|
| Breast carcinoma | N | N |
| Colonic carcinoma | S | S |
| Carcinoid | W | W |
| Gastric carcinoma | M | M |
| Leiomyosarcoma | S | S |
| Liver carcinoma | N | N |
| Lung carcinoma | N | N |
| Lymphoma | N | N |
| Melanoma | N | N |
| Mesotheiloma | N | N |
| Ovarian carcinoma | S | S |
| Pancreas carcinoma | S | S |
| Prostate carcinoma | N | N |
| Thyroid carcinoma | S | S |
| Renal cell carcinoma | N | N |
| Rhabdomyosarcoma | N | N |
| Sarcoma | S | S |
| Undifferentiated carcinoma | S | S |

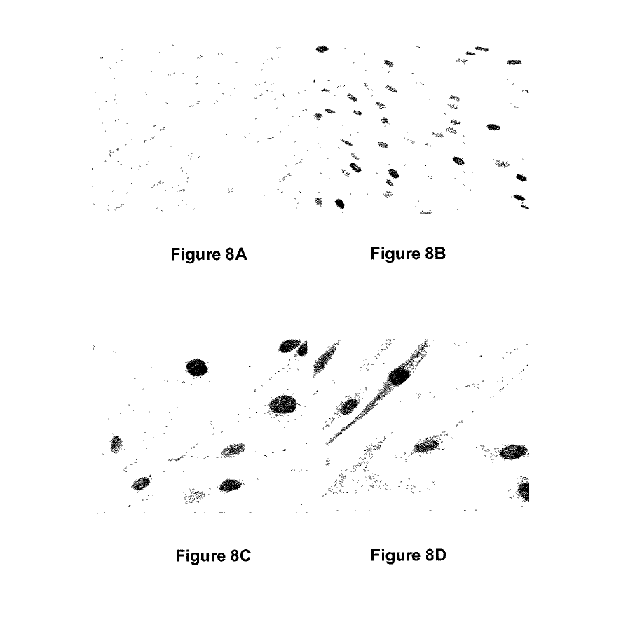

FIGS. 8-12 present some of the hyperproliferative human tissues tested for SPT1 and SPT2 expression by immunohistochemistry.

FIG. 8 shows immunolabeling of sub-confluent human dermal fibroblasts by ICC to observe the cellular expression and localization of SPT 1 and SPT2. The results for SPT1 (FIG. 8C) and SPT2 (FIG. 8D) labeled cells compared, respectively, to the negative control labeled cells (FIG. 8A) and PCNA labeled cells (FIG. 8B) show that SPT 1 and SPT2 overexpression is associated with the nucleus in proliferating fibroblasts.

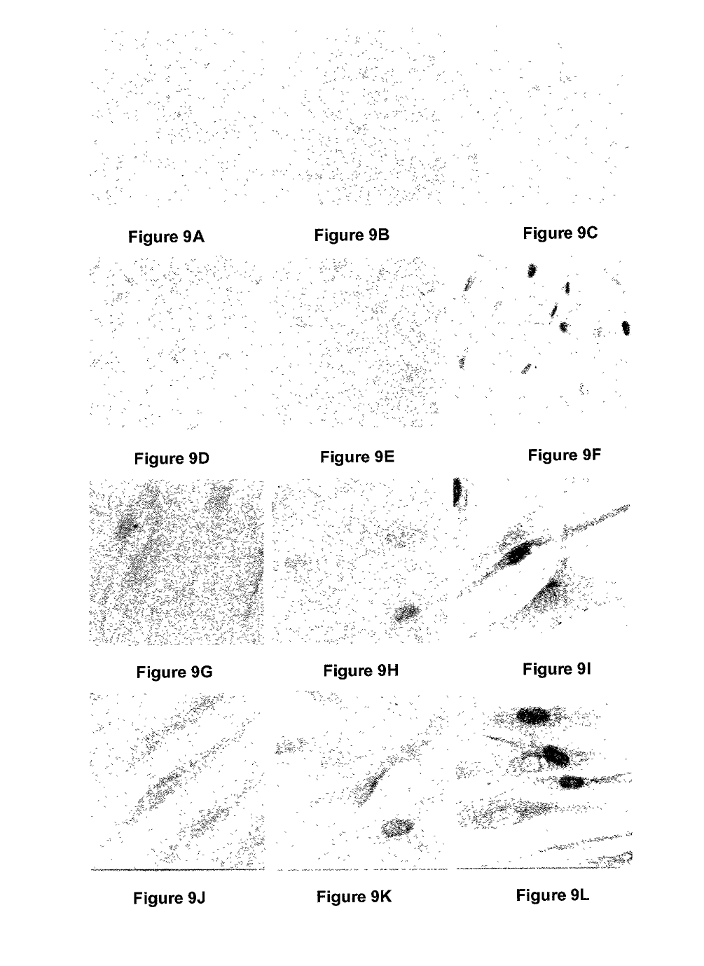

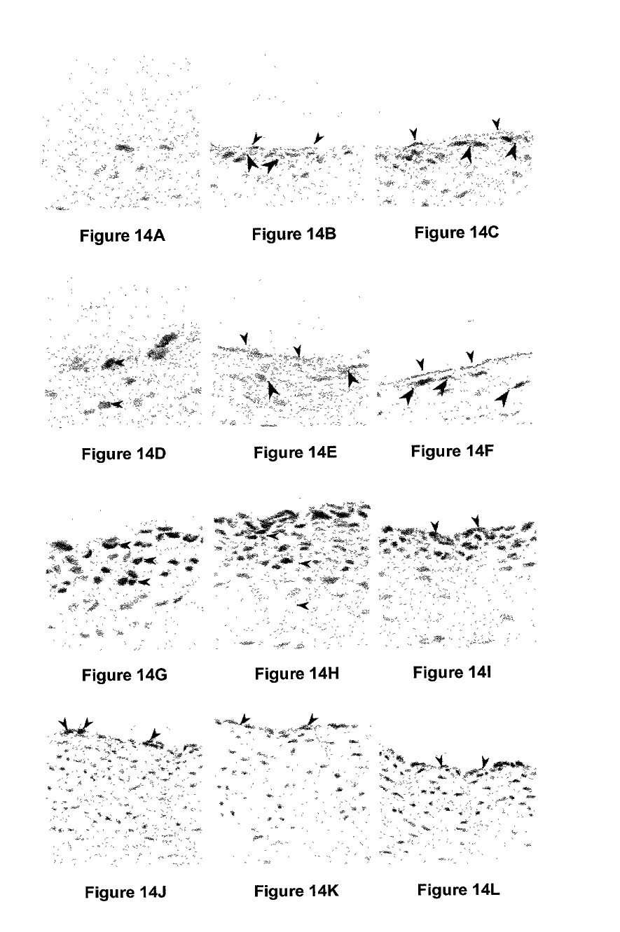

FIG. 9 shows the results of an in vitro wounding model of differentiated, quiescent fibroblasts. The model was used to mimic in vivo tissue activation to further characterize the expression of SPT in proliferating cells. Quiescent fibroblast cultures were compared to confluent cultures subjected to mechanical scraping and allowed to recover for 5 days (wound conditions). FIG. 9 shows no SPT labeling by ICC in the nine- or 14-day, quiescent cultures immunolabeled using the negative control (FIG. 9A and FIG. 9B) and PCNA (FIG. 9D and FIG. 9E) antibodies. Light diffuse labeling was observed with SPT1 (FIG. 9G and FIG. 9H) and SPT2 (FIG. 9J and FIG. 9K) antibodies. The 14 day wounded fibroblasts (FIGS. 9C, 9F, 9I and 9L) show strong immunolabeling for PCNA (FIG. 9F), SPT1 (FIG. 9I) and SPT2 (FIG. 9L). The most dramatic observation in the SPT1 and SPT2 immunolabeled cells is the intense nuclear-associated labeling in the 14 day wounded fibroblasts (FIGS. 9I and 9L) which is not present in the 9 day quiescent cells (FIGS. 9G and 9J) and 14 day quiescent cells (FIGS. 9H and 9K).

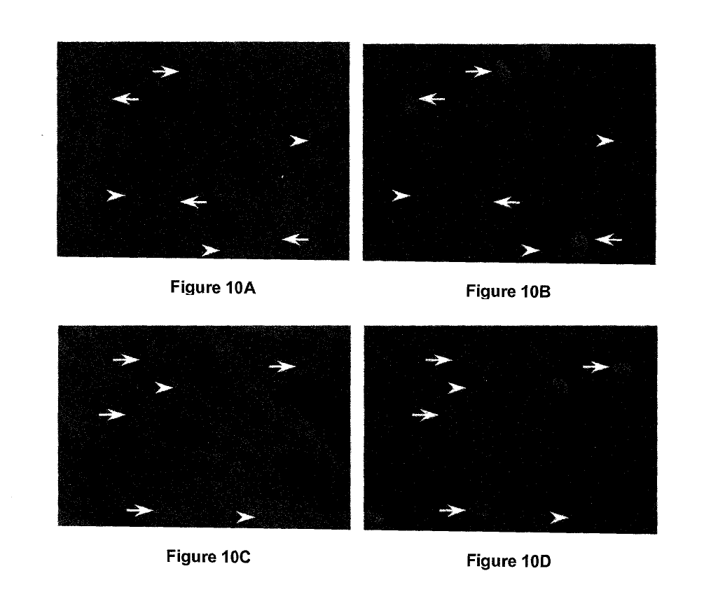

FIG. 10 shows nuclei double staining (IF:IF) used to show coincidence of PCNA (FIGS. 10B and 10D) with increased SPT1 (FIG. 10A) and SPT2 (FIG. 10C) expression. Arrows indicate cells in which PCNA was detected. The labeled cells which expressed SPT are associated with the nucleus and show strong SPT expression. Arrowheads indicate cells lacking PCNA-labeling, with diffuse SPT-labeling that does not show specific association with the nucleus. From the expression patterns observed in nuclei double staining and the in vitro wounding model, SPT expression appears to be increased in proliferating cells, with expression of SPT1 and SPT2 nuclear associated. The increased expression of SPT1 and SPT2 in de-differentiated fibroblasts and proliferating vascular smooth muscle cells in balloon-injured rat carotid artery has also been recently reported (Uhlinger D J, Carton J M, Argentieri D C and Damiano B P, R. DAM Increased Expression of Serine Palmitoyltransferase (SPT) in Balloon-injured Rat Carotid Artery, Thrombosis and Haemostasis, 2001, 86:1220-6).

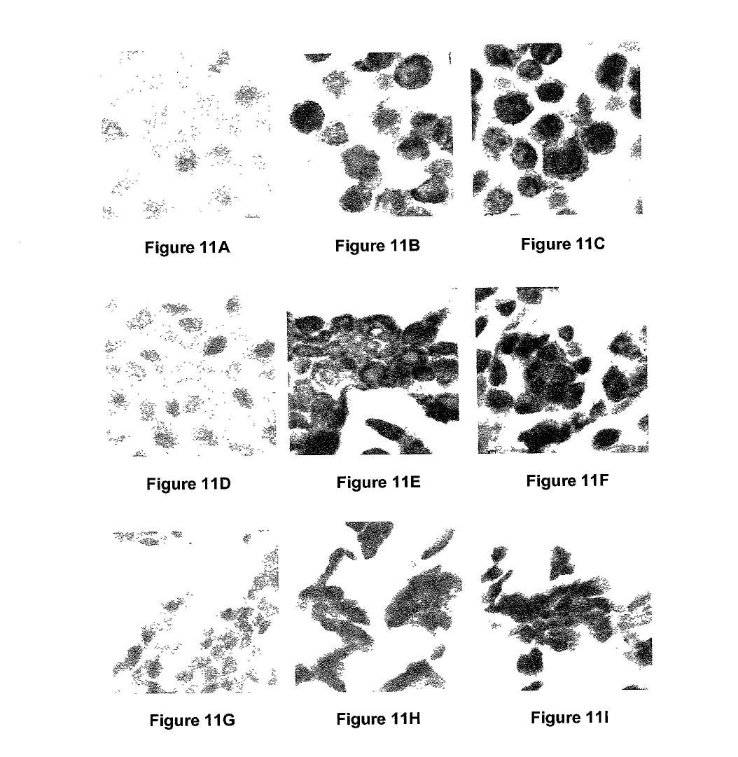

FIG. 11 shows intense SPT immunolabeling in three well-established cancer cell lines, Jurkat (FIGS. 11A, 11B and 11C), HT-29 (FIGS. 11D, 11E and 11F) and SH-SY 5Y (FIGS. 11G, 11H and 11I). The increased expression of SPT1 and SPT2 observed in both in vitro and in vivo wound repair models demonstrate parallels between wound repair responses and tumor formation. For the several cancer cell lines screened for over-expression of SPT1 and SPT2 using ICC, the results shown in Table 4 for 5 carcinomas (colon, ovarian, pancreas, thyroid and undifferentiated) and two sarcomas demonstrated that there was strong over expression of both SPT subunits; other tumors expressed little or no detectable levels of SPT 1 or SPT2.

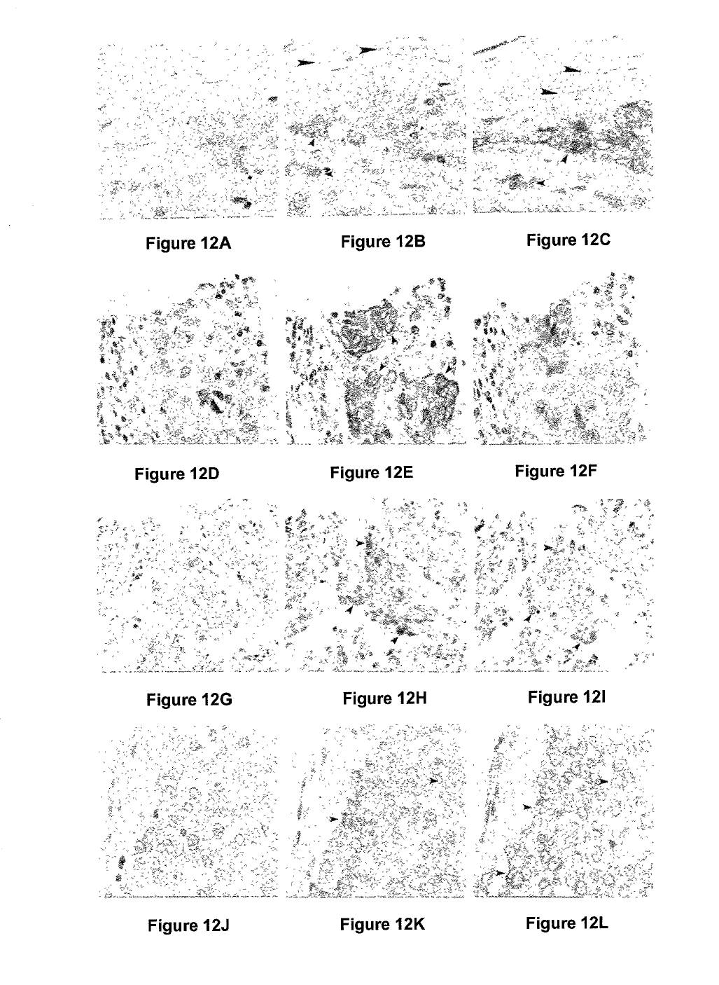

FIG. 12 shows human malignant colonic carcinoma tissues processed using IHC and antibodies to the pre-immune serum (FIG. 12A), SPT1 (FIG. 12B) and SPT2 (FIG. 12C). Strong intracellular labeling of SPT1 (small arrowheads) and SPT2 (small arrowheads) was observed in the malignant cells. Additionally, immunostaining was observed in the stromal fibroblasts adjacent to the tumor (large arrowheads). Human malignant undifferentiated carcinoma tissues (FIGS. 12D, 12E and 12F) were processed using IHC and antibodies to the pre-immune serum (FIG. 12D), SPT1 (FIG. 12E) and SPT2 (FIG. 12F). There appeared to be a strong SPT1 signal (arrowheads, FIG. 12E) in the undifferentiated human carcinoma tissue relative to the weaker SPT2 signal (FIG. 12F). Comparisons of the intensity of the signal among the tissue samples may be relevant. Since the enzymatic activity roles played by the two SPT subunits (whether catalytic or regulatory) is still unclear, it is not evident whether enhanced SPT1 expression or the ratio of SPT1 to SPT2 expression is critical to a cellular response. FIGS. 12G, 12H and 12I show human malignant thyroid carcinoma tissues, processed using antibodies to the pre-immune serum (FIG. 12G), SPT1 (FIG. 12H) and SPT2 (FIG. 12I). SPT-specific staining appeared in the majority of the cells of the tumor. The intensity of the SPT1 and SPT2 signal (arrowheads) was variable from cell to cell. Some of the malignant cells expressed very high levels of the SPT subunits, indicating that various expression levels of the subunits may be required in a tumor cell. FIGS. 12J, 12K and 12L show human malignant sarcoma tissues. SPT1 and SPT2 (arrowheads) were abundantly expressed in this malignancy.

These results indicate that the overexpression of SPT in cancer cells is not limited to those derived from epithelial cells such as carcinomas. Cancers such as sarcomas, which are derived from mesenchymal cells, also appear to have increased expression of the SPT subunits.

Results

The antibodies to human SPT1 and SPT2 developed in the present invention have enabled a method for observing the expression of these enzyme subunits in normal human tissue and elevated expression levels in vascular smooth muscle cells and activated fibroblasts in balloon injured rat carotid arteries and the proliferating cells of wounded human dermal fibroblasts demonstrated increased expression of SPT1 and SPT2 in an in vitro wounding model, showing distinct cellular up-regulation of both SPT subunits and intense immunolabeling. Quiescent, non-proliferating fibroblasts showed only light, diffuse SPT1 and SPT2 staining through out the cells. It was also apparent that a significant amount of the increased SPT1 and SPT2 immunolabeling was associated with the nucleus. It is possible that the nuclear localization of SPT is involved in sphingolipid nuclear signaling (e.g. for mitogenesis). The observation that sphingosine kinase translocates to the nucleus in fibroblasts treated with PDGF supports the hypothesis of sphingolipid involvement in nuclear signaling in proliferating and transformed cells.

The method of the present invention has enabled a means for comparing SPT expression in normal human tissues and hyperproliferative human tissues and demonstrated support for the emerging paradigm that some of the key molecules involved in the cellular wound repair response are also involved in tumor growth and metastasis.

The SPT subunits were abundantly expressed in several well-established, human tumor cell lines including a lymphoma, adenocarcinoma and a neuroblastoma. The present method has provided morphological evidence for increased expression of SPT1 and SPT2 in malignant carcinoma cells as well as in the cell types forming the tumor microenvironment, such as the reactive stromal fibroblasts and local macrophages. SPT1 and SPT2 were not detected in stromal fibroblasts in similar normal tissues. The elevated levels of the SPT subunits in these cell types suggest a role for SPT in cell metastasis activities and proliferation.

Enhanced expression of SPT in activated leukocytes involved in an inflammatory response has also been observed (2). The upregulation of SPT in human macrophages has been demonstrated to increase flux through the sphingolipid biosynthetic pathway. The increased expression of SPT in human malignancies may be indicative of regulation of flux through the sphingolipid biosynthetic pathway occurring in cells undergoing neoplastic transformation and in the cells of the surrounding microenvironment. Sphingolipids have been implicated in proliferative and metastatic processes. Changes in expression of various glycosphingolipids on the cell surface have been correlated with acquiring and maintaining cancer phenotypes, tumor progression, and metastasis.

The method of the present invention also provides the first in situ histological comparison of the expression of SPT1 and SPT2 protein in human malignant tumor cells, local macrophages and in the “reactive” stromal fibroblasts surrounding the tumor cells. The elevated SPT levels suggest possible mechanisms for some of the aberrant cellular activities within the tumor as well as within the cell types forming the tumor microenvironment (TME). The observed shift in subcellular localization of the SPT subunits in proliferating fibroblasts from diffuse and cytoplasmic to nuclear-associated suggests a functional role for this enzyme. In addition, SPT levels and cellular location may correlate with specific tumor types and the relative amounts of SPT1 and SPT2 in the tumor cells and in the stromal fibroblasts may be clinically relevant. SPT1 and SPT2 in the TME cells may be a valid predictor of metastatic activity and, thereby, have diagnostic and prognostic value. More importantly, these data suggest that the instant method may be further used as a screening method for identifying novel SPT inhibitor compounds with therapeutic utility against certain neoplasias.

### Example 4

Detection of SPT1 and SPT2 in Endothelial Tissue

Balloon Angioplasty Rat Model

Vascular injury was induced by balloon-catheter inflation of the rat common carotid artery using previously described methods. Male, Sprague Dawley rats, weighing 350-450 μm, were anesthetized with ketamine/xylazine (75/5 mg/kg, i.m.). Using aseptic techniques, a 2F embolectomy catheter (Baxter Healthcare, Irvine, Calif.) was inserted into the left common carotid via the external carotid. The balloon tip was advanced to the aorta, inflated to 30 p.s.i and slowly withdrawn with a twisting motion. This was repeated a total of three times. The catheter was removed and the external carotid was securely tied. One, 3, 7 and 14 days after injury, rats were anesthetized with ketamine/xylazine (75/5 mg/kg, i.m.). One mL of a 5% Evan's blue solution containing 1000 U Heparin was administered intravenously. Ten minutes later, rats were perfused through the aorta with saline at 100 mmHg for 10 min, followed by 4% paraformaldehyde in phosphate buffered saline (PBS). Left and right common carotids were removed and prepared for paraffin embedding. Carotids with complete thrombotic occlusion as well as carotids not stained blue in the injured segment were excluded from analysis.

Tissue Preparation

Tissue sections (5 μM) were mounted onto slides and representative sections from the middle of the carotid artery were stained with hematoxylin and eosin. Adjacent or near-adjacent sections were histochemically stained for elastin and collagen using a modified elastin-van Gieson stain (Sigma, St. Louis, Mo.). Other adjacent or near adjacent sections were used for immunohistochemical analysis.

Double Immunohistochemisty Labeling

Sections of injured and normal carotid arteries were immunohistochemically labeled with polyclonal peptide-specific antibodies raised against human SPT1 and in separate experiments with anti-peptide antibodies against human SPT2, as described in Example 2. Sections were also labeled with antibodies to factor VIII-related antigen, smooth muscle actin and a PCNA. A series of controls were performed to insure proper interpretation of labeling. Primary antibodies were substituted with the proper species isotype to control for the detection systems. On another set of controls, the first primary was omitted and the second primary antibody was processed, and vice versa.

For simultaneous double immunohistochemical labeling (IHC:IHC) each immunohistochemical labeling procedure was performed sequentially on the same section and then counter-stained. The second antigen was detected by an alkaline phosphatase-FAST RED (Dako) system. Following the first immunohistochemical labeling, the primary antibody for the second label was placed on the slides for 30 minutes at room temperature. After brief washing, the secondary biotinylated antibody was added for 30 minutes at room temperature. The slides were then washed in PBS and streptavidin-alkaline phosphatase reagent (Vector Labs) placed on the slides for 30 minutes at room temperature. After washing, slides were exposed to FAST RED CHROMOGEN (Dako). The slides were then counterstained with hematoxylin, washed, and coverslipped in a water-based mounting media (Dako) for viewing under a light microscope.

In normal vessels, SPT labeling was diffuse and patchy in medial smooth muscle and endothelium. At one and three days after injury, before appearance of neointima, SPT1 and SPT2 labeling increased in cells adjacent to damaged or necrotic smooth muscle cells. In addition, proliferating adventitial myofibroblasts labeled strongly for SPT1 and SPT2. At 7 and 14 days after injury, the media and neointima of injured vessels had increased SPT labeling which was most intense at the luminal edge of the neointima. This luminal edge has been described and shown to comprise actively proliferating smooth muscle cells. Double immunohistochemical labeling confirmed the greatest expression of SPT in areas with the greatest density of PCNA-positive cells.

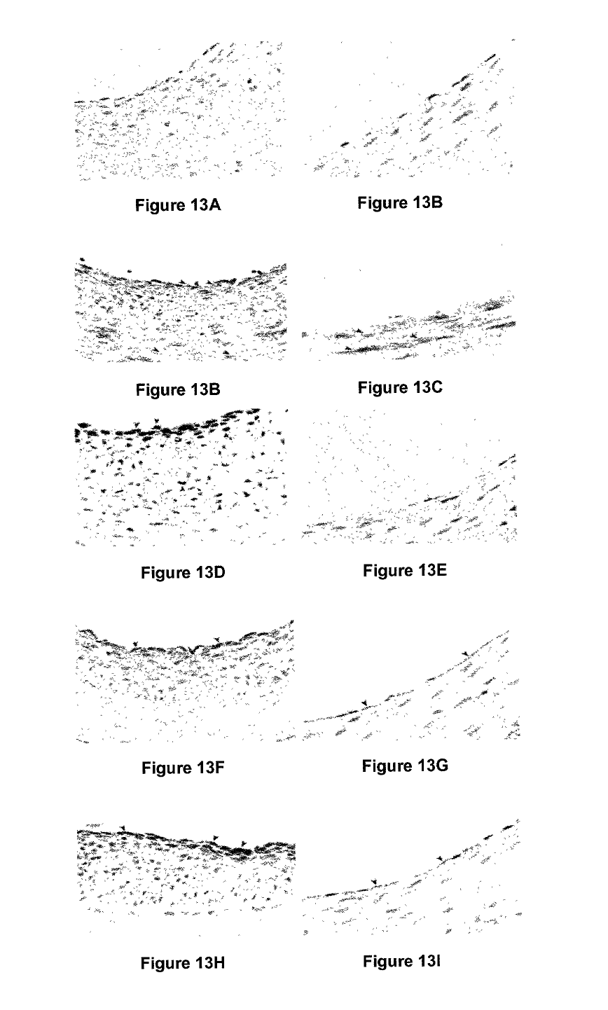

FIG. 13 shows a typical lesion observed in the rat model at 14 days after balloon angioplasty. Marked medial thickening as well as the presence of a prominent neointima in the balloon-injured artery (left panels) was observed compared to the uninjured artery (right panels). Smooth muscle actin label was present throughout the media and neointima of the injured artery as well as the media of the uninjured artery (FIG. 13C). PCNA-positive cells were observed in the media and neointima of the injured vessel with the greatest labeling at the luminal edge (FIG. 13E, arrowheads). The uninjured vessel did not express many PCNA positive cells (FIG. 13F). SPT labeling in the uninjured carotid arteries was apparent only along the intact endothelial layer (arrowheads, FIGS. 13H and 13J). At 14 days after arterial injury, SPT1 and SPT2 labeling extended from the luminal cells down through the neointima. SPT2 labeling seemed to be more intense, especially at the luminal edge. These data demonstrate that there is a specific upregulation of SPT1 and SPT2 in response to vascular injury. The upregulation of SPT1 and SPT2 is observed in cells associated with medial smooth muscle damage, proliferating adventitial myofibroblasts and smooth muscle cells of the neointima, particularly those at the proliferating luminal edge of the neointima.

FIG. 14 shows the time course of vascular injury response by examining PCNA, SPT1 and SPT2 expression at days 1, 3, 7 and 3 months after angioplasty. At day 3, PCNA labeling was apparent in smooth muscle cells (see arrows) of the injured media (FIG. 14D). SPT1 (FIG. 14E) and SPT2 (FIG. 14F) positive immunoreactivity were observed in the media (large arrowheads) as well as in the platelets deposited along the luminal edge (small arrowheads) of the injured vessels at day 3. At day 7, PCNA labeling became much more pronounced. Labeling was most intense in the early neointima but also present in the media of injured vessels (FIG. 14G). Intense SPT1 (FIG. 14H) and SPT2 (FIG. 141) immunolabeling was localized in the neointima of the injured vessels as compared to the light SPT1 and SPT2 immunolabeling in the media at day 7. Thus, the expression of SPT appears to be coincident with actively proliferating smooth muscle cells. At 3 months after injury, the labeling for PCNA, SPT1 and SPT2 is restricted to the penultimate layer at the luminal edge of the neointima.

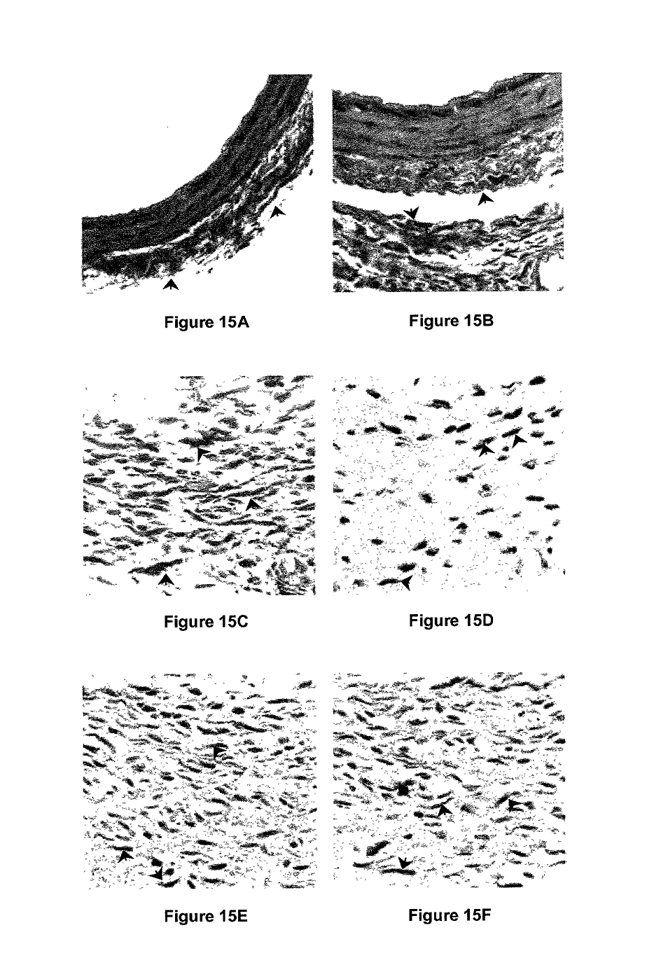

FIG. 15 shows the dedifferentiation and proliferation of myofibroblasts in the adventitia and matrix remodeling in response to the angioplasty. After hematoxylin and eosin staining of an uninjured carotid (FIG. 15A) and a 3-day injured vessel (FIG. 15B), the adventitia in the injured vessels was thicker and more cellular than that of the control vessel adventitia. Immunohistochemistry characterization was used to show the response in the adventitia of the injured vessel and antibodies to SMA (FIG. 15C), PCNA (FIG. 15D), SPT1 (FIG. 15E) and SPT2 (FIG. 15F). SMA-positive immunolabeling indicated the presence of dedifferentiated, reactive myofibroblasts in the adventitia, which was not observed in the control vessels. Prominent PCNA-positive immunolabeling (arrowheads) was also observed in the adventitia (FIG. 15D) confirming the presence of reactive fibroblasts. Prominent SPT1 (FIG. 15E) and SPT2 (FIG. 15F). Immunolabeling was also observed in myofibroblasts within the adventitia (arrowheads). These characteristic wound response changes were similar to that observed in the variety of carcinomas and indicate that SPT may be involved in signaling pathways common to both hyperplasia and neoplasia of fibroblasts.

### Example 5

In Vivo Restenosis Model

Male, Sprague Dawley rats, weighing 350-450 gm, are anesthetized with ketamine/xylazine (75/5 mg/kg, i.m.). Vascular injury is induced by balloon-catheter inflation of the rat common carotid artery using methods that have been previously described (Damiano et al., 1999). One group of rats is treated with 0.5 mg/kg of the serine palmitoyltransferase inhibitor myriocin injected i.p. at Day 0, 2, 5 and 10. Animals are sacrificed on Day 1, 3, 7 and 14 to assess histopathology of carotid restenotic injury and to examine the expression of the serine palmitoyltransferase subunits immunohistochemically in sections from the injured and control. A decrease in the extent of the restenotic injury (i.e. narrowing of the vessel) in the treated animals is an indication of efficacy of the SPT inhibitor.

### Example 6

B16 Lung Metastasis Model

B16-F10 mouse melanoma cells (ICLC catalog code ATL99010) are grown as monolayer tissue cultures using standard conditions. Approximately 2×10⁶ cells are injected iv. into the tail vein of 4 to 8 week old C57BL/6 mice. In one group of mice a serine palmitoyltransferase inhibitor such as myriocin is simultaneously administered ip at a concentration of about 0.5 mg/kg at Day 0, Day 2, and Day 5. Control groups are injected with a solvent vehicle. The mice are maintained for 9 days post tumor injection to allow the tumors to establish and then are euthanized. The lungs are removed and fixed in Bouin's solution and the number of lung metastasis are counted using a dissecting microscope. A decrease in the number of lung metastasis is an indication that the SPT inhibitor prevented establishment of the B16-F10 tumor in the mice.

### Example 7

Detection of SPT2 in Inflamed Colon—Rat Colitis Model

The animals were anesthetized with halothane and their colons were washed with ethanol (30%, 1 ml, approximately 30 s) to break the mucous barrier, followed by a saline rinse (1 ml). Either zymosan (1 ml, 25 mg/ml, Sigma, St. Louis, Mo.) or an equal volume of vehicle (saline) was then instilled into the colon through a gavage needle inserted intra-anally to a depth of about 7-8 cm. The zymosan animals were sacrificed 20 hours following intracolonic instillation. The animals were transcardially perfused with fixative. The colons were removed and post-fixed in the same solution.

Tissue Preparation

From the distal end of the colon, an area known to have colitis response in this model. approximately 3 consecutive 1 cm sections of the colon were cut and were embedded in one paraffin block per animal to allow simultaneous observations. Tissue sections (5 μM) were cut from each paraffin block. Sections were mounted onto slides and were processed for immunohistochemical analysis using antibodies specific to SPT1 and SPT2. Additional immunohistochemical markers were used to verify the presence of polymorphonuclear leukocytes (PMNs) using an antibody specific to myeloperoxidase and macrophages using an antibody MAC-1 specific for macrophages.

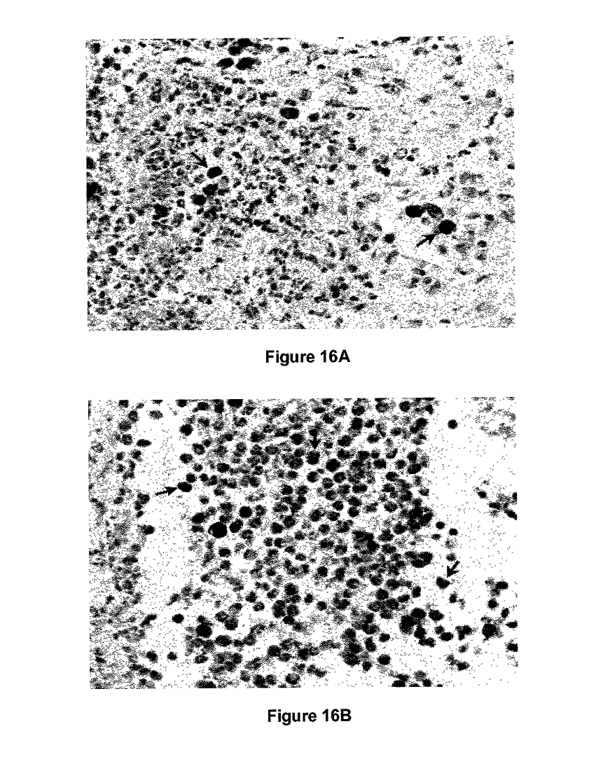

FIG. 16 illustrates the leukocyte infiltration into the inflamed colon. The arrows in the upper panel (FIG. 16A) indicate activated macrophages with enhanced levels of SPT2. The lower panel (FIG. 16B) shows a neutrophil infiltrate on the lumenal aspect of the inflamed colon. The arrows indicate three of the numerous activated neutrophils in the field displaying enhanced SPT2 expression. Similar results were obtained for the SPT1 immunolabeling (data not shown). In addition to the marked presence of SPT1 and SPT2 immunopositive PMNs and macrophages, positive immunolabeling on the migrating epithelial tongue was observed, which appeared to be migrating from the normal epithelium (data not shown), suggesting that SPT1 and SPT2 are not only present in the inflammatory cells but are also in the proliferating epithelial cells (perhaps in an attempt to resolve the wound).

While the foregoing specification teaches the principles of the present invention, with examples provided for the purposes of illustration, it will be understood that further modifications of various aspects of the invention will be apparent to those skilled in the art in view of this description and that practice of the invention encompasses all of the usual variations, alternative embodiments, adaptations and/or modifications as come within the scope of the following claims and their equivalents, which are to be interpreted to embrace all such variations.

What is claimed is:

1. A method for measuring the expression level of serine palmitoyltransferase by mammalian cells comprising:
(a) contacting a serine palmitoyltransferase specific compound with a mammalian cell to form a plurality of compound-serine palmitoyltransferase complexes; and,
(b) measuring the level of serine palmitoyltransferase expressed by the cell by detecting the presence of the complexes.

2. A method for measuring the expression of serine palmitoyltransferase by mammalian cells comprising:
(a) contacting a serine palmitoyltransferase specific compound with a first mammalian cell which basally expresses serine palmitoyltransferase to form a first plurality of compound-serine palmitoyltransferase complexes;
(b) contacting a serine palmitoyltransferase specific compound with a second mammalian cell which hyperproliferatively expresses serine palmitoyltransferase to form a second plurality of compound-serine palmitoyltransferase complexes;
(c) determining the levels of serine palmitoyltransferase expressed by the first and second cells by detecting the presence of the first and second plurality of complexes; and
(d) measuring the difference in the levels of serine palmitoyltransferase expressed by the first and second cells.

3. A method for in vivo imaging of a tissue comprising:
(a) administering to a mammal a serine palmitoyltransferase specific compound, wherein the compound comprises a detectable label, and wherein the compound binds to mammalian cells which hyperproliferatively express serine palmitoyltransferase; and,
(b) determining the location of the cells within a tissue of the mammal by imaging the detectable label.

4. The method of claim 2 further comprising the step of using the measured difference in the levels of serine palmitoyltransferase expressed by the first and second cells to detect, diagnose, diagnose the metastatic potential of, monitor the prognosis and progression of, or monitor the therapeutic efficacy of a treatment of a cancer.

5. The method of claim 4, wherein the cancer is selected from the group consisting of breast carcinoma, colonic carcinoma, carcinoid, gastric carcinoma, glioma, hepatoma, leiomyosarcoma, liver carcinoma, lung carcinoma, lymphoma, melanoma, mesothelioma, myeloma, ovarian carcinoma, pancreas carcinoma, prostate carcinoma, thyroid carcinoma, renal cell carcinoma, retinoblastoma, rhabdomyosarcoma, sarcoma, undifferentiated carcinoma, and leukemia.

6. The method of claim 2 further comprising the step of using the measured difference in levels of serine palmitoyltransferase expressed by the first and second cells to detect or diagnose the occurrence of vascular injury.

7. The method of claim 6 wherein the vascular injury is restenosis.

8. The method of claim 2 further comprising the step of using the measured difference in levels of serine palmitoyltransferase expressed by the first and second cells to detect or diagnose the occurrence of inflammation.

9. The method of claim 8 wherein the inflammation is the result of ulcerative colitis, inflammatory bowel syndrome, Crohn's Disease, rheumatoid arthritis, atherosclerosis, stroke, or asthma.

10. A method for screening a therapeutically effective compound that inhibits serine palmitoyltransferase comprising:
(a) contacting a serine palmitoyltransferase specific compound with a first mammalian cell which hyperproliferatively expresses serine palmitoyltransferase to form a first plurality of compound-serine palmitoyltransferase complexes;
(b) contacting a potential serine palmitoyltransferase inhibitor compound with a second mammalian cell which hyperproliferatively expresses serine palmitoyltransferase to form a second plurality of compound-serine palmitoyltransferase complexes;
(c) determining the levels of serine palmitoyltransferase expressed by the first and second cells by detecting the presence of the first and second plurality of compound-serine palmitoyltransferase complexes; and,
(d) measuring the difference in the levels of serine palmitoyltransferase expressed by the first and second cells to determine whether the potential inhibitor compound inhibits serine palmitoyltransferase expression.

11. The method of claim 1, wherein the serine palmitoyltransferase specific compound is selected from the group consisting of a compound that binds to serine palmitoyltransferase, a monospecific antibody that binds to serine palmitoyltransferase, and a nucleic acid that will hybridize with serine palmitoyltransferase mRNA.

12. The method of claim 2, wherein the serine palmitoyltransferase specific compound is selected from the group consisting of a compound that binds to serine palmitoyltransferase, a monospecific antibody that binds to serine palmitoyltransferase, and a nucleic acid that will hybridize with serine palmitoyltransferase mRNA.

13. The method of claim 3, wherein the serine palmitoyltransferase specific compound is selected from the group consisting of a compound that binds to serine palmitoyltransferase, a monospecific antibody that binds to serine palmitoyltransferase, and a nucleic acid that will hybridize with serine palmitoyltransferase mRNA.

14. The method of claim 10, wherein the serine palmitoyltransferase specific compound is selected from the group consisting of a compound that binds to serine palmitoyltransferase, a monospecific antibody that binds to serine palmitoyltransferase, and a nucleic acid that will hybridize with serine palmitoyltransferase mRNA.

15. The method of claim 1 wherein the mammalian cells which express serine palmitoyltransferase are selected from the group consisting of adrenal cells, brain cells, breast cells, colon cells, epithelial cells, endothelial cells, heart cells, immunological cells, kidney cells, liver cells, lung cells, ovary cells, pancreas cells, prostate cells, skin cells, spleen cells, stomach cells, testis cells, thyroid cells, uterus cells and vascular cells.

16. The method of claim 15 wherein the epithelial cells are selected from the group consisting of endothelial cells, non-glial neuronal cells, colon cells, breast cells, the proximal tubules of the kidney, smooth muscle of the prostate, smooth muscle of the uterus and smooth muscle of the testis.

17. The method of claim 15 wherein the immunological cells are selected from the group consisting of polymorphonuclear leukocytes, monocytes, macrophages, epitheloid cells, giant cells, microglia, Kupffer cells and alveolar macrophages.

18. The method of claim 2 wherein the mammalian cells which basally express serine palmitoyltransferase are selected from the group consisting of adrenal cells, brain cells, breast cells, colon cells, epithelial cells, endothelial cells, heart cells, immunological cells, kidney cells, liver cells, lung cells, ovary cells, pancreas cells, prostate cells, skin cells, spleen cells, stomach cells, testis cells, thyroid cells, uterus cells, and vascular cells.

19. The method of claim 2 wherein the mammalian cells which hyperproliferatively express serine palmitoyltransferase are selected from the group consisting of epithelial cells, endothelial cells, cancer cells, immunological cells, and cells within a tumor microenvironment.

20. The method of claim 19, wherein the cancer cells are selected from the group consisting of breast carcinoma, colonic carcinoma, carcinoid, gastric carcinoma, glioma, hepatoma, leiomyosarcoma, liver carcinoma, lung carcinoma, lymphoma, melanoma, mesothelioma, myeloma, ovarian carcinoma, pancreas carcinoma, prostate carcinoma, thyroid carcinoma, renal cell carcinoma, retinoblastoma, rhabdomyosarcoma, sarcoma, undifferentiated carcinoma, and leukemia.

21. The method of claim 19, wherein the epithelial cells are selected from the group consisting of endothelial cells, non-glial neuronal cells, colon cells, breast cells, the proximal tubules of the kidney, smooth muscle of the prostate, smooth muscle of the uterus and smooth muscle of the testis.

22. The method of claim 19 wherein the immunological cells are selected from the group consisting of polymorphonuclear leukocytes, monocytes, macrophages, epitheloid cells, giant cells, microglia, Kupffer cells and alveolar macrophages.

23. The method of claim 19 wherein the cells within a tumor microenvironment are selected from the group consisting of stromal fibroblasts, stromal monocytes and myofibroblasts.

24. A method for treating a serine palmitoyltransferase mediated disorder in a subject in need thereof comprising administering a therapeutically effective amount of a pharmaceutical formulation of a serine palmitoyltransferase inhibitor compound to the subject; wherein, optionally, the serine palmitoyltransferase inhibitor compound is cytotoxic.

25. The method of claim 24, wherein the serine palmitoyltransferase inhibitor compound is a serine palmitoyltransferase specific compound.

26. The method of claim 24, wherein the serine palmitoyltransferase mediated disorder is selected from the group consisting of cancer, inflammation, and vascular injury.

27. The method of claim 26 wherein the cancer is selected from the group consisting of breast carcinoma, colonic carcinoma, carcinoid, gastric carcinoma, glioma, hepatoma, leiomyosarcoma, liver carcinoma, lung carcinoma, lymphoma, melanoma, mesothelioma, myeloma, ovarian carcinoma, pancreas carcinoma, prostate carcinoma, thyroid carcinoma, renal cell carcinoma, retinoblastoma, rhabdomyosarcoma, sarcoma, undifferentiated carcinoma, and leukemia.

28. The method of claim 26 wherein the inflammation is the result of ulcerative colitis, inflammatory bowel syndrome, Crohn's Disease, rheumatoid arthritis, atherosclerosis, stroke, or asthma.

29. The method of claim 26 wherein the vascular injury is the result of restenosis.

30. The method of claim 25 wherein the serine palmitoyltransferase specific compound is selected from the group consisting of a monospecific antibody optionally labeled with a cytotoxic agent, and a nucleic acid that will optionally hybridize to serine palmitoyltransferase mRNA.

31. The method of claim 24 wherein the pharmaceutical formulation is coated onto a balloon catheter or stent and released in a time-dependent manner.
